SEQUENCE LISTING (24 sequences)

SEQ ID NO: 1 - DNA, 798 bp, synthetic construct
  misc_feature 1..798: note = Synthetic: HPV genotype 16 consensus E6-E7 DNA sequence
  source 1..798: mol_type = other DNA
atggactggacctggatcctgttcctggtggccgccgccacacgggtgcacagcttccaggacccccaggagagcggcagaaagctgcctcagctgtgtaccgagctgcagaccaccatccacgacatcatcctggagtgtgtgtactgtaagcagcagctgctgaggagagaggtgtacgaccgggacctgtgtatcgtgtacagggacggcaatccctacgccgtgtgtgacaagtgcctgaagttctacagcaagatcagcgagtaccggcactactgctacagcctgtacggcaccaccctggagcagcagtacaacaagcccctgtgtgacctgctgatccggtgtatcaactgccagaagcccctgcagagacacctggacaagaagcagcggttccacaacatcaggggcagatggaccggcagatgtatgagctgctgccggagcagcagaaccagaagggagacccagctgagaggccggaagagaagaagccacggcgatacccccaccctgcacgagtacatgctggacctgcagcctgagaccaccgatctgtacggctacggccagctgaatgacagcagcgaggaggaggatgagatcgacggccctgccggccaggccgagcccgacagagcccactacaacatcgtgaccttttgctgtaagtgtgacagcaccctgagactgtgcgtgcagagcacccacgtggacatcagaaccctggaggatctgctgatgggcaccctgggcatcgtgtgtcccatctgctcccagaaaccctgatga

SEQ ID NO: 2 - protein, 264 aa, synthetic construct
  REGION 1..264: note = Synthetic: HPV genotype 16 consensus E6-E7 protein sequence
MDWTWILFLVAAATRVHSFQDPQESGRKLPQLCTELQTTIHDIILECVYCKQQLLRREVYDRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQLRGRKRRSHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 3 - protein, 15 aa, Human papillomavirus 16
  REGION 1..15: note = misc_feature - HPV16 E6 immunodominant epitope
LCIVYRDGNPYAVCD

SEQ ID NO: 4 - protein, 15 aa, Human papillomavirus 16
  REGION 1..15: note = misc_feature - HPV16 E7 immunodominant epitope
AEPDRAHYNIVTFCC

SEQ ID NO: 5 - protein, 142 aa, synthetic construct
  REGION 1..142: note = Synthetic: HPV16 E6 consensus sequence
FQDPQESGRKLPQLCTELQTTIHDIILECVYCKQQLLRREVYDRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQL

SEQ ID NO: 6 - protein, 97 aa, synthetic construct
  REGION 1..97: note = Synthetic: HPV16 E7 consensus sequence
HGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 7 - protein, 18 aa, synthetic construct
  REGION 1..18: note = Synthetic: IgE Leader Sequence
MDWTWILFLVAAATRVHS

SEQ ID NO: 8 - protein, 7 aa, synthetic construct
  REGION 1..7: note = Synthetic: Proteolytic Cleavage Sequence
RGRKRRS

SEQ ID NO: 9 - DNA, 783 bp, synthetic construct
  misc_feature 1..783: note = Synthetic: HPV18 Consensus Sequence encoding E6 and E7
  source 1..783: mol_type = other DNA
gccagattcgaggaccccaccaggagcggctacaagctgcccgatctgtgtaccgagctgaacaccagcctgcaggacatcgagatcacctgtgtgtactgtaagaccgtgctggagctgaccgaggtgttcgagaaggacctgttcgtggtgtacagggacagcatcccccacgccgcctgccacaagtgtatcgacttctacagccggatccgggagctgagacactacagcgacagcgtgtacggcgataccctggagaagctgaccaacaccggcctgtacaacctgctgatccggtgcctgagatgccagaagcccctgctgagacacctgaacgagaagcggcggttccacaacatcgccggccactacagaggccagtgccacagctgctgtaacagggccaggcaggagagactgcagcggagaagagagacccaggtgaggggcaggaagagaagaagccacggccccaaggccaccctgcaggatatcgtgctgcacctggagccccagaatgagatccccgtggatctgctgggccacggccagctgtccgacagcgaggaggagaacgacgagatcgacggcgtgaatcaccagcacctgcctgccagaagagccgagcctcagaggcacaccatgctgtgtatgtgctgtaagtgtgaggcccggatcgaactggtggtggagagcagcgccgacgacctgagagccttccagcagctgttcctgaacaccctgagcttcgtgtgtccttggtgtgccagccagcagtga

SEQ ID NO: 10 - protein, 260 aa, synthetic construct
  REGION 1..260: note = Synthetic: HPV18 Consensus HPV peptide sequence with E6 and E7
ARFEDPTRSGYKLPDLCTELNTSLQDIEITCVYCKTVLELTEVFEKDLFVVYRDSIPHAACHKCIDFYSRIRELRHYSDSVYGDTLEKLTNTGLYNLLIRCLRCQKPLLRHLNEKRRFHNIAGHYRGQCHSCCNRARQERLQRRRETQVRGRKRRSHGPKATLQDIVLHLEPQNEIPVDLLGHGQLSDSEEENDEIDGVNHQHLPARRAEPQRHTMLCMCCKCEARIELVVESSADDLRAFQQLFLNTLSFVCPWCASQQ

SEQ ID NO: 11 - DNA, 54 bp, synthetic construct
  misc_feature 1..54: note = Synthetic: IgE Leader DNA sequence
  source 1..54: mol_type = other DNA
atggactggacctggatcctgttcctggtggccgctgccacacgggtgcacagc

SEQ ID NO: 12 - protein, 18 aa, synthetic construct
  REGION 1..18: note = Synthetic: IgE leader peptide sequence
MDWTWILFLVAAATRVHS

SEQ ID NO: 13 - DNA, 837 bp, synthetic construct
  misc_feature 1..837: note = Synthetic: HPV18 Consensus Sequence encoding E6 and E7; include IgE leader
  source 1..837: mol_type = other DNA
atggactggacctggatcctgttcctggtggccgctgccacacgggtgcacagcgccagattcgaggaccccaccaggagcggctacaagctgcccgatctgtgtaccgagctgaacaccagcctgcaggacatcgagatcacctgtgtgtactgtaagaccgtgctggagctgaccgaggtgttcgagaaggacctgttcgtggtgtacagggacagcatcccccacgccgcctgccacaagtgtatcgacttctacagccggatccgggagctgagacactacagcgacagcgtgtacggcgataccctggagaagctgaccaacaccggcctgtacaacctgctgatccggtgcctgagatgccagaagcccctgctgagacacctgaacgagaagcggcggttccacaacatcgccggccactacagaggccagtgccacagctgctgtaacagggccaggcaggagagactgcagcggagaagagagacccaggtgaggggcaggaagagaagaagccacggccccaaggccaccctgcaggatatcgtgctgcacctggagccccagaatgagatccccgtggatctgctgggccacggccagctgtccgacagcgaggaggagaacgacgagatcgacggcgtgaatcaccagcacctgcctgccagaagagccgagcctcagaggcacaccatgctgtgtatgtgctgtaagtgtgaggcccggatcgaactggtggtggagagcagcgccgacgacctgagagccttccagcagctgttcctgaacaccctgagcttcgtgtgtccttggtgtgccagccagcagtga

SEQ ID NO: 14 - protein, 278 aa, synthetic construct
  REGION 1..278: note = Synthetic: HPV18 Consensus HPV peptide sequence with E6 and E7; including IgE leader
MDWTWILFLVAAATRVHSARFEDPTRSGYKLPDLCTELNTSLQDIEITCVYCKTVLELTEVFEKDLFVVYRDSIPHAACHKCIDFYSRIRELRHYSDSVYGDTLEKLTNTGLYNLLIRCLRCQKPLLRHLNEKRRFHNIAGHYRGQCHSCCNRARQERLQRRRETQVRGRKRRSHGPKATLQDIVLHLEPQNEIPVDLLGHGQLSDSEEENDEIDGVNHQHLPARRAEPQRHTMLCMCCKCEARIELVVESSADDLRAFQQLFLNTLSFVCPWCASQQ

SEQ ID NO: 15 - DNA, 3824 bp, synthetic construct
  misc_feature 1..3824: note = Synthetic: pGX3002 plasmid with consensus and IgE leader
  source 1..3824: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatggactggacctggatcctgttcctggtggccgctgccacacgggtgcacagcgccagattcgaggaccccaccaggagcggctacaagctgcccgatctgtgtaccgagctgaacaccagcctgcaggacatcgagatcacctgtgtgtactgtaagaccgtgctggagctgaccgaggtgttcgagaaggacctgttcgtggtgtacagggacagcatcccccacgccgcctgccacaagtgtatcgacttctacagccggatccgggagctgagacactacagcgacagcgtgtacggcgataccctggagaagctgaccaacaccggcctgtacaacctgctgatccggtgcctgagatgccagaagcccctgctgagacacctgaacgagaagcggcggttccacaacatcgccggccactacagaggccagtgccacagctgctgtaacagggccaggcaggagagactgcagcggagaagagagacccaggtgaggggcaggaagagaagaagccacggccccaaggccaccctgcaggatatcgtgctgcacctggagccccagaatgagatccccgtggatctgctgggccacggccagctgtccgacagcgaggaggagaacgacgagatcgacggcgtgaatcaccagcacctgcctgccagaagagccgagcctcagaggcacaccatgctgtgtatgtgctgtaagtgtgaggcccggatcgaactggtggtggagagcagcgccgacgacctgagagccttccagcagctgttcctgaacaccctgagcttcgtgtgtccttggtgtgccagccagcagtgatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 16 - DNA, 738 bp, synthetic construct
  misc_feature 1..738: note = Synthetic: HPV genotype 16 consensus E6-E7 peptide sequence without IgE leader coding sequence
  source 1..738: mol_type = other DNA
ttccaggacccccaggagagcggcagaaagctgcctcagctgtgtaccgagctgcagaccaccatccacgacatcatcctggagtgtgtgtactgtaagcagcagctgctgaggagagaggtgtacgaccgggacctgtgtatcgtgtacagggacggcaatccctacgccgtgtgtgacaagtgcctgaagttctacagcaagatcagcgagtaccggcactactgctacagcctgtacggcaccaccctggagcagcagtacaacaagcccctgtgtgacctgctgatccggtgtatcaactgccagaagcccctgcagagacacctggacaagaagcagcggttccacaacatcaggggcagatggaccggcagatgtatgagctgctgccggagcagcagaaccagaagggagacccagctgagaggccggaagagaagaagccacggcgatacccccaccctgcacgagtacatgctggacctgcagcctgagaccaccgatctgtacggctacggccagctgaatgacagcagcgaggaggaggatgagatcgacggccctgccggccaggccgagcccgacagagcccactacaacatcgtgaccttttgctgtaagtgtgacagcaccctgagactgtgcgtgcagagcacccacgtggacatcagaaccctggaggatctgctgatgggcaccctgggcatcgtgtgtcccatctgctcccagaaaccc

SEQ ID NO: 17 - DNA, 426 bp, synthetic construct
  misc_feature 1..426: note = Synthetic: HPV genotype 16 consensus E6 nucleotide sequence
  source 1..426: mol_type = other DNA
ttccaggacccccaggagagcggcagaaagctgcctcagctgtgtaccgagctgcagaccaccatccacgacatcatcctggagtgtgtgtactgtaagcagcagctgctgaggagagaggtgtacgaccgggacctgtgtatcgtgtacagggacggcaatccctacgccgtgtgtgacaagtgcctgaagttctacagcaagatcagcgagtaccggcactactgctacagcctgtacggcaccaccctggagcagcagtacaacaagcccctgtgtgacctgctgatccggtgtatcaactgccagaagcccctgcagagacacctggacaagaagcagcggttccacaacatcaggggcagatggaccggcagatgtatgagctgctgccggagcagcagaaccagaagggagacccagctg

SEQ ID NO: 18 - DNA, 21 bp, synthetic construct
  misc_feature 1..21: note = Synthetic: Nucleotide sequence encoding Proteolytic Cleavage Sequence
  source 1..21: mol_type = other DNA
agaggccggaagagaagaagc

SEQ ID NO: 19 - DNA, 291 bp, synthetic construct
  misc_feature 1..291: note = Synthetic: HPV genotype 16 consensus E7 nucleotide sequence
  source 1..291: mol_type = other DNA
cacggcgatacccccaccctgcacgagtacatgctggacctgcagcctgagaccaccgatctgtacggctacggccagctgaatgacagcagcgaggaggaggatgagatcgacggccctgccggccaggccgagcccgacagagcccactacaacatcgtgaccttttgctgtaagtgtgacagcaccctgagactgtgcgtgcagagcacccacgtggacatcagaaccctggaggatctgctgatgggcaccctgggcatcgtgtgtcccatctgctcccagaaaccc

SEQ ID NO: 20 - protein, 246 aa, synthetic construct
  REGION 1..246: note = Synthetic: HPV genotype 16 consensus E6-E7 peptide sequence without IgE leader sequence
FQDPQESGRKLPQLCTELQTTIHDIILECVYCKQQLLRREVYDRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQLRGRKRRSHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 21 - protein, 149 aa, synthetic construct
  REGION 1..149: note = Synthetic: HPV18 Consensus E6 peptide
ARFEDPTRSGYKLPDLCTELNTSLQDIEITCVYCKTVLELTEVFEKDLFVVYRDSIPHAACHKCIDFYSRIRELRHYSDSVYGDTLEKLTNTGLYNLLIRCLRCQKPLLRHLNEKRRFHNIAGHYRGQCHSCCNRARQERLQRRRETQV

SEQ ID NO: 22 - protein, 104 aa, synthetic construct
  REGION 1..104: note = Synthetic: HPV18 Consensus E7 peptide
HGPKATLQDIVLHLEPQNEIPVDLLGHGQLSDSEEENDEIDGVNHQHLPARRAEPQRHTMLCMCCKCEARIELVVESSADDLRAFQQLFLNTLSFVCPWCASQQ

SEQ ID NO: 23 - DNA, 447 bp, synthetic construct
  misc_feature 1..447: note = Synthetic: HPV18 Consensus Sequence encoding E6
  source 1..447: mol_type = other DNA
gccagattcgaggaccccaccaggagcggctacaagctgcccgatctgtgtaccgagctgaacaccagcctgcaggacatcgagatcacctgtgtgtactgtaagaccgtgctggagctgaccgaggtgttcgagaaggacctgttcgtggtgtacagggacagcatcccccacgccgcctgccacaagtgtatcgacttctacagccggatccgggagctgagacactacagcgacagcgtgtacggcgataccctggagaagctgaccaacaccggcctgtacaacctgctgatccggtgcctgagatgccagaagcccctgctgagacacctgaacgagaagcggcggttccacaacatcgccggccactacagaggccagtgccacagctgctgtaacagggccaggcaggagagactgcagcggagaagagagacccaggtg

SEQ ID NO: 24 - DNA, 312 bp, synthetic construct
  misc_feature 1..312: note = Synthetic: HPV18 Consensus Sequence encoding E7
  source 1..312: mol_type = other DNA
cacggccccaaggccaccctgcaggatatcgtgctgcacctggagccccagaatgagatccccgtggatctgctgggccacggccagctgtccgacagcgaggaggagaacgacgagatcgacggcgtgaatcaccagcacctgcctgccagaagagccgagcctcagaggcacaccatgctgtgtatgtgctgtaagtgtgaggcccggatcgaactggtggtggagagcagcgccgacgacctgagagccttccagcagctgttcctgaacaccctgagcttcgtgtgtccttggtgtgccagccagcag

(19) United States
(12) Patent Application Publication
(10) Pub. No.: US 2023/0233659 A1
(43) Pub. Date: Jul. 27, 2023

(54) COMPOSITIONS AND METHODS FOR TREATING ANAL HIGH-GRADE SQUAMOUS INTRAEPITHELIAL LESION (HSIL)

(71) Applicant: Inovio Pharmaceuticals, Inc., Plymouth Meeting, PA (US)

(72) Inventors: Jian Yan, Wallingford, PA (US); Jong Joseph Kim, Blue Bell, PA (US); Prakash Bhuyan, Lafayette Hill, PA (US); Jeffrey Skolnik, Cherry Hill, NJ (US)

(21) Appl. No.: 18/048,776

(22) Filed: Oct. 21, 2022

Related U.S. Application Data
(60) Provisional application No. 63/270,929, filed on Oct. 22, 2021.

(51) Int. Cl. A61K 39/12; A61K 9/00; A61P 17/02

(52) U.S. Cl. CPC A61K 39/12; A61K 9/0019; A61P 17/02

(57) ABSTRACT

The use of anti-HPV immunogens and nucleic acid molecules that encode them for the treatment and prevention of anal high-grade squamous intraepithelial lesion are disclosed. Pharmaceutical composition, recombinant vaccines comprising DNA plasmid and live attenuated vaccines are disclosed, as well as methods of inducing an immune response to treat or prevent anal high-grade squamous intraepithelial lesion.

### CROSS-REFERENCE TO RELATED APPLICATIONS

This application claims the benefit of U.S. Provisional Patent Application No. 63/270,929, filed Oct. 22, 2021, the disclosure of which is incorporated herein by reference in their entirety.

### FIELD OF THE INVENTION

The present invention relates to improved vaccines, improved methods for inducing immune responses, and for prophylactically and/or therapeutically immunizing individuals against anal high-grade squamous intraepithelial lesion (HSIL).

### BACKGROUND OF THE INVENTION

Anal squamous intraepithelial lesions include both low-grade squamous intraepithelial lesions (LSIL) and high-grade squamous intraepithelial lesions (HSIL) and are caused by chronic infection with the human papillomavirus (HPV). The current management of anal high grade squamous intraepithelial lesions (HSIL) is challenging and there is significant evidence that untreated HSIL can progress to squamous cell carcinoma.

Thus, there is a need in the art for improved compositions and methods for treatment or prevention of anal HSIL. The present invention satisfies this unmet need.

### SUMMARY OF THE INVENTION

Aspects of the invention provide compositions comprising at least one nucleotide sequence comprising an HPV16 E6-E7 fusion antigen, an HPV18 E6-E7 fusion antigen, or a combination thereof; and uses thereof for the treatment or prevention of anal high-grade squamous intraepithelial lesion.

Another aspect provides compositions comprising one or more nucleotide sequences encoding an HPV16 E6-E7 fusion antigen selected from the group consisting of: nucleotide sequence that encodes SEQ ID NO:2; a nucleotide sequence that is at least 95% homologous to a nucleotide sequence that encodes SEQ ID NO:2; a nucleotide sequence that is at least 95% homologous to a fragment of a nucleotide sequence that encodes SEQ ID NO:2. In some embodiments, the nucleotide sequences encoding the HPV6 E6-E7 fusion antigen are without a leader sequence at 5′ end.

In another aspect of the invention, there are provided compositions comprising one or more nucleotide sequences encoding an HPV16 E6-E7 fusion antigen selected from the group consisting of: SEQ ID NO:1; a nucleotide sequence that is at least 95% homologous to SEQ ID NO:1; a fragment of SEQ ID NO:1; a nucleotide sequence that is at least 95% homologous to a fragment of SEQ ID NO:1. In some embodiments, the nucleotide sequences encoding the HPV16 E6-E7 fusion antigen are without a leader sequence at 5′ end.

Another aspect provides compositions comprising one or more nucleotide sequences encoding an HPV18 E6-E7 fusion antigen selected from the group consisting of: nucleotide sequence that encodes SEQ ID NO:10; a nucleotide sequence that is at least 95% homologous to a nucleotide sequence that encodes SEQ ID NO:10; a nucleotide sequence that is at least 95% homologous to a fragment of a nucleotide sequence that encodes SEQ ID NO:10. In some embodiments, the nucleotide sequences encoding the HPV6 E6-E7 fusion antigen are further comprises a nucleotide encoding a leader sequence at the 5′ end.

In another aspect of the invention, there are provided compositions comprising one or more nucleotide sequences encoding an HPV18 E6-E7 fusion antigen selected from the group consisting of: SEQ ID NO:9; a nucleotide sequence that is at least 95% homologous to SEQ ID NO:9; a fragment of SEQ ID NO:9; a nucleotide sequence that is at least 95% homologous to a fragment of SEQ ID NO:9. In some embodiments, the nucleotide sequences encoding the HPV16 E6-E7 fusion antigen further comprises a nucleotide encoding a leader sequence at the 5′ end.

The nucleotide sequences provided can be a plasmid.

In additional aspects, provided are pharmaceutical compositions comprising the disclosed nucleotide sequences.

In some aspects, there are methods of treating or preventing anal high-grade squamous intraepithelial lesion in an individual by inducing an effective immune response in an individual, comprising administering to said individual a composition comprising one or more of the nucleotides sequences provided. The methods preferably include a step of introducing the provided nucleotide sequences into the individual by electroporation.

### BRIEF DESCRIPTION OF THE FIGURES

FIG. 1 depicts the study design of the present study.

FIG. 2 provides data on the efficacy of the vaccine at weeks 36 and 64.

FIG. 3 provides data demonstrating that VGX-3100 induces a cellular immune response to both HPV16 and HPV18 antigens.

FIG. 4 provides data demonstrating that VGX-3100 induces a humoral immune response to HPV16E7 and HPV18E7 antigens.

FIG. 5 provides data on the safety of the VGX-3100 vaccine.

### DETAILED DESCRIPTION OF PREFERRED EMBODIMENTS

### Definitions

The disclosed methods may be understood more readily by reference to the following detailed description, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods, which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the term “at least one” means “one or more.”

As used herein, the term “subject” or “individual” as used herein refers to any animal, but in particular humans. Thus, the methods are applicable to human and nonhuman animals, although preferably used most preferably with humans. “Subject” and “patient” and “individual” are used interchangeably herein.

As used herein, the term “comprising” is intended to include examples encompassed by the terms “consisting essentially of” and “consisting of”; similarly, the term “consisting essentially of” is intended to include examples encompassed by the term “consisting of.”

“Adjuvant” as used herein may mean any molecule added to the DNA plasmid vaccines described herein to enhance antigenicity of the one or more antigens encoded by the DNA plasmids and encoding nucleic acid sequences described hereinafter.

“Antibody” may mean an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antigen” refers to: proteins having an HPV E6 or HPV E7 domain, and preferably and E6 and E7 fusion with an endoproteolytic cleavage site therebetween. Antigens include SEQ ID NO: 2 (subtype 16) and SEQ ID NO: 4 (subtype 18); fragments thereof of lengths set forth herein, variants, i.e. proteins with sequences homologous to SEQ ID NO:2 or SEQ ID NO:4 as set forth herein, fragments of variants having lengths set forth herein, and combinations thereof. Antigens may have an IgE leader sequence of SEQ ID NO:7 or 12 or may alternatively have such sequence removed from the N-terminal end. Antigens may optionally include signal peptides such as those from other proteins.

“Biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extent the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Coding sequence” or “encoding nucleic acid” as used herein may mean refers to the nucleic acid (RNA or DNA molecule) that comprise a nucleotide sequence which encodes an antigen as set forth in section c. above. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides, e.g., an IgE leader sequence such as SEQ ID NO:7 or 12.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Fragment” may mean a polypeptide fragment of an antigen that is capable of eliciting an immune response in a mammal against the antigen. A fragment of an antigen may be 100% identical to the full length except missing at least one amino acid from the N and/or C terminal, in each case with or without signal peptides and/or a methionine at position 1. Fragments may comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full-length antigen, excluding any heterologous signal peptide added. The fragment may, preferably, comprise a fragment of a polypeptide that is 95% or more, 96% or more, 97% or more, 98% or more or 99% or more homologous to the antigen and additionally comprise an N terminal methionine or heterologous signal peptide which is not included when calculating percent homology Fragments may further comprise an N terminal methionine and/or a signal peptide such as an immunoglobulin signal peptide, for example an IgE or IgG signal peptide. The N terminal methionine and/or signal peptide may be linked to a fragment of an antigen.

A fragment of a nucleic acid sequence that encodes antigen may be 100% identical to the full length except missing at least one nucleotide from the 5′ and/or 3′ end, in each case with or without sequences encoding signal peptides and/or a methionine at position 1. Fragments may comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may, preferably, comprise a fragment that encodes a polypeptide that is 95% or more, 96% or more, 97% or more, 98% or more or 99% or more homologous to the antigen and additionally optionally comprise sequence encoding an N terminal methionine or heterologous signal peptide which is not included when calculating percent homology Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Immune response” as used herein may mean the activation of a host's immune system, e.g., that of a mammal, in response to the introduction of one or more antigens via the provided DNA plasmid vaccines. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

As used herein, the term “placebo” means administration of a pharmaceutical composition that does not include VGX-3100.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5 10° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

As used herein, “treating” and like terms refer to reducing the severity and/or frequency of human papillomavirus (HPV) type 16- or HPV type 18-related high grade anal or anal/peri-anal intraepithelial lesion (HSIL) of the anus and/or peri-anus symptoms for example and anal or anal/peri-anal high grade squamous intraepithelial lesions (HSIL) lesions; eliminating HPV type 16 or HPV type 18 infection symptoms, especially HSIL lesions; and/or clearing HPV type 16 or HPV type 18 virus from the subject; and/or resolution to anal or anal/peri-anal low grade squamous intraepithelial lesions (LSIL) or normal tissue.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

### Description

Improved vaccines are disclosed which arise from a multi-phase strategy to enhance cellular immune responses induced by immunogens. Modified consensus sequences were generated. Genetic modifications including codon optimization, RNA optimization, and the addition of a high efficient immunoglobin leader sequence are also disclosed. The novel construct has been designed to elicit stronger and broader cellular immune responses than corresponding codon-optimized immunogens.

The improved HPV vaccines are based upon proteins and genetic constructs that encode proteins with epitopes that make them particularly effective as immunogens, such that they mediate a prophylactic or therapeutic strategy against anal high-grade squamous intraepithelial lesion (HSIL). Accordingly, vaccines may induce a therapeutic or prophylactic immune response. In some embodiments, the means to deliver the immunogen is a DNA vaccine, a recombinant vaccine, a protein subunit vaccine, a composition comprising the immunogen, an attenuated vaccine or a killed vaccine. In some embodiments, the vaccine comprises a combination selected from the groups consisting of: one or more DNA vaccines, one or more recombinant vaccines, one or more protein subunit vaccines, one or more compositions comprising the immunogen, one or more attenuated vaccines and one or more killed vaccines.

According to some embodiments, a vaccine is delivered to an individual to modulate the activity of the individual's immune system and thereby enhance the immune response against HPV to treat anal high-grade squamous intraepithelial lesion. When a nucleic acid molecule that encodes the protein is taken up by cells of the individual the nucleotide sequence is expressed in the cells and the protein are thereby delivered to the individual. Methods of delivering the coding sequences of the protein on nucleic acid molecule such as plasmid, as part of recombinant vaccines and as part of attenuated vaccines, as isolated proteins or proteins part of a vector are provided.

Compositions and methods are provided which provide a prophylactic and/or therapeutic treatment against anal high-grade squamous intraepithelial lesion in an individual.

Compositions for delivering nucleic acid molecules that comprise a nucleotide sequence that encodes the immunogen are operably linked to regulatory elements. Compositions may include a plasmid that encodes the immunogen, a recombinant vaccine comprising a nucleotide sequence that encodes the immunogen, a live attenuated pathogen that encodes a protein of the invention and/or includes a protein of the invention; a killed pathogen includes a protein of the invention; or a composition such as a liposome or subunit vaccine that comprises a protein of the invention. The present invention further relates to injectable pharmaceutical compositions that comprise compositions.

Aspects of the invention provide compositions comprising at least one nucleotide sequence encoding at least one HPV E6-E7 fusion antigen, for example an HPV16 E6-E7 fusion antigen or an HPV18 E6-E7 fusion antigen. In one embodiment, the composition comprises a nucleotide sequence encoding an HPV16 E6-E7 fusion antigen and an HPV18 E6-E7 fusion antigen.

In one embodiment, the invention include methods of administrating the composition of the invention into a subject in need thereof. In one embodiment, the subject is a subject diagnosed with anal high-grade squamous intraepithelial lesion. In one embodiment, the subject is subject having anal high-grade squamous intraepithelial lesion. In one embodiment, the subject is a subject at risk of developing anal high-grade squamous intraepithelial lesion.

### HPV16 E6-E7 Fusion

Another aspect provides compositions comprising one or more nucleotide sequences encoding an HPV16 E6-E7 fusion antigen selected from the group consisting of: nucleotide sequence that encodes SEQ ID NO:2; a nucleotide sequence that is at least 95% homologous to a nucleotide sequence that encodes SEQ ID NO:2; a fragment of a nucleotide sequence that encodes SEQ ID NO:2; a nucleotide sequence that is at least 95% homologous to a fragment of a nucleotide sequence that encodes SEQ ID NO:2.

In some embodiments the compositions include HPV16 E6-E7 fusion antigens selected from the group consisting of: nucleotide sequence that encodes SEQ ID NO:2; a nucleotide sequence that is at least 95% homologous to a nucleotide sequence that encodes SEQ ID NO:2; a fragment of a nucleotide sequence that encodes SEQ ID NO:2; a nucleotide sequence that is at least 95% homologous to a fragment of a nucleotide sequence that encodes SEQ ID NO:2.

In another aspect of the invention, there are provided compositions comprising one or more nucleotide sequences encoding an HPV16 E6-E7 fusion antigen selected from the group consisting of: SEQ ID NO:1; a nucleotide sequence that is at least 95% homologous to SEQ ID NO:1; a fragment of SEQ ID NO:1; a nucleotide sequence that is at least 95% homologous to a fragment of SEQ ID NO:1.

In some embodiments the nucleotide sequences described herein is absent the leader sequence. In one embodiment, the nucleotide sequences comprising HPV16 E6-E7 fusion antigen is absent a leader sequence. In particular, the HPV16 E6-E7 fusion antigens including nucleotide sequence that encodes SEQ ID NO:2; are absent a leader sequence at 5′ end, for example nucleotide sequence encoding SEQ ID NO:7. In particular, the HPV6 E6-E7 fusion antigens including nucleotide sequence SEQ ID NO:1 are absent a leader sequence at 5′ end, for example nucleotide sequence encoding SEQ ID NO:7.

In some embodiments the nucleotide sequences of the present invention can be 80%, 81%, 82%, 83%, 84%, 85%, 86%, 87%, 88%, 89%, 90%, 91%, 92%, 93%, 94%, 95%, 96%, 97%, 98%, or 99% homologous with the provided nucleotide sequences; preferably 95%, 96%, 97%, 98%, or 99%; or 98% or 99%.

The nucleotide sequences provided can be included into one of a variety of known vectors or delivery systems, including a plasmid, viral vector, lipid vector, nanoparticle; preferably a plasmid.

In additional aspects, provided are pharmaceutical compositions comprising the disclosed nucleotide sequences.

In some aspects, there are methods of inducing an effective immune response in an individual against more than one subtype of HPV thereby providing a prophylactic or therapeutic treatment against anal high-grade squamous intraepithelial lesion, comprising administering to said individual a composition comprising one or more of the nucleotides sequences provided; preferably, the compositions have more than one antigen. The methods preferably include a step of introducing the provided nucleotide sequences into the individual by electroporation.

SEQ ID NO:1 comprises a nucleotide sequence that encodes a consensus immunogen of HPV16 E6 and E7 proteins, that comprises and IgE leader sequence, a consensus sequence for HPV E6, linked to a consensus sequence for HPV E7 by a proteolytic cleavage sequence. SEQ ID NO: 2 comprises the amino acid sequence of a consensus immunogen of HPV16 E6 and E7 proteins, that comprises and IgE leader sequence, a consensus sequence for HPV E6, linked to a consensus sequence for HPV E7 by a proteolytic cleavage sequence. The consensus sequence for HPV16 E6 includes the immunodominant epitope set forth in SEQ ID NO:3. The consensus sequence for HPV16 E7 includes the immunodominant epitope set forth in SEQ ID NO:4. The consensus sequence for HPV E6 is SEQ ID NO:5. The consensus sequence for HPV E6 is SEQ ID NO:6. The IgE leader sequence is SEQ ID NO:7. A proteolytic cleavage sequence useful to link the two consensus sequences is SEQ ID NO:8.

Further information regarding the HPV16 E6-E7 fusion antigen can be found at least in U.S. Pat. No. 8,168,769, which is incorporated by reference in its entirety.

In some embodiments, vaccines include SEQ ID NO:2, or a nucleic acid molecule that encodes SEQ ID NO:2. In some embodiments, vaccines of the invention include SEQ ID NO:3 and/or SEQ ID NO:4, or nucleic acid sequence which encode one of both of them. In some embodiments, vaccines of the invention include SEQ ID NO:5 and/or the SEQ ID NO:6, or nucleic acid sequences which encode one or both of them. In some embodiments, vaccines of the invention include SEQ ID NO:5 linked to SEQ ID NO:6 by a proteolytic cleavage sequence such as SEQ ID NO:8, or nucleic acid sequence which encodes the fusion protein. In some embodiments, vaccines of the present invention include the IgE leader sequence SEQ ID NO:7 or nucleic acid sequence which encodes the same. In some embodiments, vaccines of the invention include SEQ ID NO:2 or the nucleic acid sequence in SEQ ID NO:1.

Fragments of SEQ ID NO:2 may be 100% identical to the full length except missing at least one amino acid from the N and/or C terminal, in each case with or without signal peptides and/or a methionine at position 1. Fragments of SEQ ID NO:2 can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the full length SEQ ID NO:2, excluding any heterologous signal peptide added. The fragment can, preferably, comprise a fragment of SEQ ID NO:2 that is 95% or more, 96% or more, 97% or more, 98% or more or 99% or more homologous to SEQ ID NO:2 and additionally comprise an N terminal methionine or heterologous signal peptide which is not included when calculating percent homology Fragments can further comprise an N terminal methionine and/or a signal peptide such as an immunoglobulin signal peptide, for example an IgE or IgG signal peptide. The N terminal methionine and/or signal peptide may be linked to the fragment.

Fragments of a nucleic acid sequence SEQ ID NO:1 can be 100% identical to the full length except missing at least one nucleotide from the 5′ and/or 3′ end, in each case with or without sequences encoding signal peptides and/or a methionine at position 1. Fragments can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of full length coding sequence SEQ ID NO:1, excluding any heterologous signal peptide added. The fragment can, preferably, comprise a fragment that encodes a polypeptide that is 95% or more, 96% or more, 97% or more, 98% or more or 99% or more homologous to the antigen SEQ ID NO:2 and additionally optionally comprise sequence encoding an N terminal methionine or heterologous signal peptide which is not included when calculating percent homology Fragments can further comprise coding sequences for an N terminal methionine and/or a signal peptide such as an immunoglobulin signal peptide, for example an IgE or IgG signal peptide. The coding sequence encoding the N terminal methionine and/or signal peptide may be linked to the fragment.

Fragments of SEQ ID NO:1 may comprise 30 or more nucleotides, including preferably sequences that encode an immunodominant epitope. In some embodiments, fragments of SEQ ID NO:1 may comprise 45 or more nucleotides, including preferably sequences that encode an immunodominant epitope. In some embodiments, fragments of SEQ ID NO:1 may comprise 60 or more nucleotides, including preferably sequences that encode an immunodominant epitope. In some embodiments, fragments of SEQ ID NO:1 may comprise 75 or more nucleotides, including preferably sequences that encode an immunodominant epitope. In some embodiments, fragments of SEQ ID NO:1 may comprise 90 or more nucleotides, including preferably sequences that encode an immunodominant epitope. In some embodiments, fragments of SEQ ID NO:1 may comprise 120 or more nucleotides, including preferably sequences that encode an immunodominant epitope. In some embodiments, fragments of SEQ ID NO:1 may comprise 150 or more nucleotides, including preferably sequences that encode an immunodominant epitope. In some embodiments, fragments of SEQ ID NO:1 may comprise 180 or more nucleotides, including preferably sequences that encode an immunodominant epitope. In some embodiments, fragments of SEQ ID NO:1 may comprise 210 or more nucleotides, including preferably sequences that encode an immunodominant epitope. In some embodiments, fragments of SEQ ID NO:1 may comprise 240 or more nucleotides, including preferably sequences that encode an immunodominant epitope. In some embodiments, fragments of SEQ ID NO:1 may comprise 270 or more nucleotides, including preferably sequences that encode an immunodominant epitope. In some embodiments, fragments of SEQ ID NO:1 may comprise 300 or more nucleotides, including preferably sequences that encode an immunodominant epitope. In some embodiments, fragments of SEQ ID NO:1 may comprise 360 or more nucleotides, including preferably sequences that encode an immunodominant epitope. In some embodiments, fragments of SEQ ID NO:1 may comprise 420 or more nucleotides, including preferably sequences that encode an immunodominant epitope. In some embodiments, fragments of SEQ ID NO:1 may comprise 480 or more nucleotides, including preferably sequences that encode an immunodominant epitope. In some embodiments, fragments of SEQ ID NO:1 may comprise 540 or more nucleotides, including preferably sequences that encode an immunodominant epitope. In some embodiments, fragments of SEQ ID NO:1 may comprise 600 or more nucleotides, including preferably sequences that encode an immunodominant epitope. In some embodiments, fragments of SEQ ID NO:1 may comprise 300 or more nucleotides, including preferably sequences that encode an immunodominant epitope. In some embodiments, fragments of SEQ ID NO:1 may comprise 660 or more nucleotides, including preferably sequences that encode an immunodominant epitope. In some embodiments, fragments of SEQ ID NO:1 may comprise 720 or more nucleotides, including preferably sequences that encode an immunodominant epitope. In some embodiments, fragments of SEQ ID NO:1 may comprise 780 or more nucleotides, including preferably sequences that encode an immunodominant epitope. In some embodiments, fragments of SEQ ID NO:1 may comprise coding sequences for the IgE leader sequences. In some embodiments, fragments of SEQ ID NO:1 do not comprise coding sequences for the IgE leader sequences. Fragments may comprise fewer than 60 nucleotides, in some embodiments fewer than 75 nucleotides, in some embodiments fewer than 90 nucleotides, in some embodiments fewer than 120 nucleotides, in some embodiments fewer than 150 nucleotides, in some embodiments fewer than 180 nucleotides, in some embodiments fewer than 210 nucleotides, in some embodiments fewer than 240 nucleotides, in some embodiments fewer than 270 nucleotides, in some embodiments fewer than 300 nucleotides, in some embodiments fewer than 360 nucleotides, in some embodiments fewer than 420 nucleotides, in some embodiments fewer than 480 nucleotides, in some embodiments fewer than 540 nucleotides, in some embodiments fewer than 600 nucleotides, in some embodiments fewer than 660 nucleotides, in some embodiments fewer than 720 nucleotides, and in some embodiments fewer than 780 nucleotides.

Fragments of SEQ ID NO:2 may comprise 15 or more amino acids, including preferably sequences that encode an immunodominant epitope. In some embodiments, fragments of SEQ ID NO:2 may comprise 18 or more amino acids, including preferably sequences that encode an immunodominant epitope. In some embodiments, fragments of SEQ ID NO:2 may comprise 21 or more amino acids, including preferably sequences that encode an immunodominant epitope. In some embodiments, fragments of SEQ ID NO:2 may comprise 24 or more amino acids, including preferably sequences that encode an immunodominant epitope. In some embodiments, fragments of SEQ ID NO:2 may comprise 30 or more amino acids, including preferably sequences that encode an immunodominant epitope. In some embodiments, fragments of SEQ ID NO:2 may comprise 36 or more amino acids, including preferably sequences that encode an immunodominant epitope. In some embodiments, fragments of SEQ ID NO:2 may comprise 42 or more amino acids, including preferably sequences that encode an immunodominant epitope. In some embodiments, fragments of SEQ ID NO:2 may comprise 48 or more amino acids, including preferably sequences that encode an immunodominant epitope. In some embodiments, fragments of SEQ ID NO:2 may comprise 54 or more amino acids, including preferably sequences that encode an immunodominant epitope. In some embodiments, fragments of SEQ ID NO:2 may comprise 60 or more amino acids, including preferably sequences that encode an immunodominant epitope. In some embodiments, fragments of SEQ ID NO:2 may comprise 18 or more amino acids, including preferably sequences that encode an immunodominant epitope. In some embodiments, fragments of SEQ ID NO:2 may comprise 72 or more amino acids, including preferably sequences that encode an immunodominant epitope. In some embodiments, fragments of SEQ ID NO:2 may comprise 90 or more amino acids, including preferably sequences that encode an immunodominant epitope. In some embodiments, fragments of SEQ ID NO:2 may comprise 120 or more amino acids, including preferably sequences that encode an immunodominant epitope. In some embodiments, fragments of SEQ ID NO:2 may comprise 150 or more amino acids, including preferably sequences that encode an immunodominant epitope. In some embodiments, fragments of SEQ ID NO:2 may comprise 180 or more amino acids, including preferably sequences that encode an immunodominant epitope. In some embodiments, fragments of SEQ ID NO:2 may comprise 210 or more amino acids, including preferably sequences that encode an immunodominant epitope. In some embodiments, fragments of SEQ ID NO:2 may comprise 240 or more amino acids, including preferably sequences that encode an immunodominant epitope. In some embodiments, fragments of SEQ ID NO:2 may comprise 260 or more amino acids, including preferably sequences that encode an immunodominant epitope. In some embodiments, fragments of SEQ ID NO:2 may comprise coding sequences for the IgE leader sequences. In some embodiments, fragments of SEQ ID NO:2 do not comprise coding sequences for the IgE leader sequences. Fragments may comprise fewer than 24 amino acids, in some embodiments fewer than 30 amino acids, in some embodiments fewer than 36 amino acids, in some embodiments fewer than 42 amino acids, in some embodiments fewer than 48 amino acids, in some embodiments fewer than 54 amino acids, in some embodiments fewer than 60 amino acids, in some embodiments fewer than 72 amino acids, in some embodiments fewer than 90 amino acids, in some embodiments fewer than 120 amino acids, in some embodiments fewer than 150 amino acids, in some embodiments fewer than 180 amino acids, in some embodiments fewer than 210 amino acids in some embodiments fewer than 240 amino acids, and in some embodiments fewer than 260 amino acids.

### HPV18 E6-E7 Fusion

Another aspect provides compositions comprising one or more nucleotide sequences encoding an HPV18 E6-E7 fusion antigen selected from the group consisting of: nucleotide sequence that encodes SEQ ID NO:10; a nucleotide sequence that is at least 95% homologous to a nucleotide sequence that encodes SEQ ID NO:10; a fragment of a nucleotide sequence that encodes SEQ ID NO:10; a nucleotide sequence that is at least 95% homologous to a fragment of a nucleotide sequence that encodes SEQ ID NO:10.

In some embodiments the compositions include HPV18 E6-E7 fusion antigens selected from the group consisting of: nucleotide sequence that encodes SEQ ID NO:10; a nucleotide sequence that is at least 95% homologous to a nucleotide sequence that encodes SEQ ID NO:10; a fragment of a nucleotide sequence that encodes SEQ ID NO:10; a nucleotide sequence that is at least 95% homologous to a fragment of a nucleotide sequence that encodes SEQ ID NO:10.

In another aspect of the invention, there are provided compositions comprising one or more nucleotide sequences encoding an HPV18 E6-E7 fusion antigen selected from the group consisting of: SEQ ID NO:9; a nucleotide sequence that is at least 95% homologous to SEQ ID NO:9; a fragment of SEQ ID NO:9; a nucleotide sequence that is at least 95% homologous to a fragment of SEQ ID NO:9.

In some embodiments the nucleotide sequences described herein is absent the leader sequence. In one embodiment, the nucleotide sequences comprising HPV18 E6-E7 fusion antigen is absent a leader sequence. In particular, the HPV18 E6-E7 fusion antigens including nucleotide sequence that encodes SEQ ID NO:10; are absent a leader sequence at 5′ end, for example nucleotide sequence encoding SEQ ID NO:12. In particular, the HPV16 E6-E7 fusion antigens including nucleotide sequence SEQ ID NO:9 are absent a leader sequence at 5′ end, for example nucleotide sequence comprising SEQ ID NO:11.

In some embodiments the compositions include HPV18 E6-E7 fusion antigens selected from the group consisting of: nucleotide sequence that encodes SEQ ID NO:14; a nucleotide sequence that is at least 95% homologous to a nucleotide sequence that encodes SEQ ID NO:14; a fragment of a nucleotide sequence that encodes SEQ ID NO:14; a nucleotide sequence that is at least 95% homologous to a fragment of a nucleotide sequence that encodes SEQ ID NO:14. SEQ ID NO:14 comprises the amino acid sequence of the HPV18 E6-E7 fusion antigen of SEQ ID NO:10 and further comprises an IgE leader sequence.

In another aspect of the invention, there are provided compositions comprising one or more nucleotide sequences encoding an HPV18 E6-E7 fusion antigen selected from the group consisting of: SEQ ID NO:13; a nucleotide sequence that is at least 95% homologous to SEQ ID NO:13; a fragment of SEQ ID NO:9; a nucleotide sequence that is at least 95% homologous to a fragment of SEQ ID NO:13. SEQ ID NO:13 comprises the nucleotide sequence of SEQ ID NO:9 encoding a HPV18 E6-E7 fusion antigen and further comprises a nucleotide sequence encoding an IgE leader sequence.

In some embodiments the nucleotide sequences of the present invention can be 80%, 81%, 82%, 83%, 84%, 85%, 86%, 87%, 88%, 89%, 90%, 91%, 92%, 93%, 94%, 95%, 96%, 97%, 98%, or 99% homologous with the provided nucleotide sequences; preferably 95%, 96%, 97%, 98%, or 99%; or 98% or 99%.

The nucleotide sequences provided can be included into one of a variety of known vectors or delivery systems, including a plasmid, viral vector, lipid vector, nanoparticle; preferably a plasmid.

In additional aspects, provided are pharmaceutical compositions comprising the disclosed nucleotide sequences.

In some aspects, there are methods of inducing an effective immune response in an individual against more than one subtype of HPV thereby providing a prophylactic or therapeutic treatment against anal high-grade squamous intraepithelial lesion, comprising administering to said individual a composition comprising one or more of the nucleotides sequences provided; preferably, the compositions have more than one antigen. The methods preferably include a step of introducing the provided nucleotide sequences into the individual by electroporation.

SEQ ID NO:9 comprises a nucleotide sequence that encodes a consensus immunogen of HPV18 E6 and E7 proteins. SEQ ID NO:13 includes SEQ ID NO:9 and further comprises an IgE leader sequence linked to the nucleotide sequence that encodes a consensus immunogen of HPV18 E6 and E7 proteins. SEQ ID NO:10 comprises the amino acid sequence for the consensus immunogen of HPV18 E6 and E7 proteins. SEQ ID NO:14 includes SEQ ID NO:10 and further comprises an IgE leader sequence linked to a consensus immunogen sequence. The IgE leader sequence is SEQ ID NO:12 and may be encoded by SEQ ID NO:11. SEQ ID NO:15 is the nucleic acid sequence of the plasmid pGX3002 with SEQ ID NO:13 incorporated for expression therein.

Further information regarding the HPV16 E6-E7 fusion antigen can be found at least in U.S. Pat. No. 8,389,706, which is incorporated by reference in its entirety.

In some embodiments, vaccines include SEQ ID NO:10, or a nucleic acid molecule that encodes SEQ ID NO:10. In some embodiments, vaccines include SEQ ID NO:9 as a nucleic acid molecule that encodes SEQ ID NO:10. In some embodiments, vaccines comprise SEQ ID NO:14 or a nucleic acid molecule that encodes SEQ ID NO:14. In some embodiments, vaccines comprise SEQ ID NO:13 as a nucleic acid molecule that encodes SEQ ID NO:14. In some embodiments, vaccines comprise SEQ ID NO:15.

Fragments of SEQ ID NO:10 or 14 may be 100% identical to the full length except missing at least one amino acid from the N and/or C terminal, in each case with or without signal peptides and/or a methionine at position 1. Fragments of SEQ ID NO:10 or 15 can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the full length SEQ ID NO:10 or 14, excluding any heterologous signal peptide added. The fragment can, preferably, comprise a fragment of SEQ ID NO:10 or 15 that is 95% or more, 96% or more, 97% or more, 98% or more or 99% or more homologous to SEQ ID NO:10 or 14 and additionally comprise an N terminal methionine or heterologous signal peptide which is not included when calculating percent homology. Fragments can further comprise an N terminal methionine and/or a signal peptide such as an immunoglobulin signal peptide, for example an IgE or IgG signal peptide. The N terminal methionine and/or signal peptide may be linked to the fragment.

Fragments of a nucleic acid sequence SEQ ID NO:9 or 13 can be 100% identical to the full length except missing at least one nucleotide from the 5′ and/or 3′ end, in each case with or without sequences encoding signal peptides and/or a methionine at position 1. Fragments can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of full length coding sequence SEQ ID NO:9 or 13, excluding any heterologous signal peptide added. The fragment can, preferably, comprise a fragment that encodes a polypeptide that is 95% or more, 96% or more, 97% or more, 98% or more or 99% or more homologous to the antigen SEQ ID NO:10 or 14 and additionally optionally comprise sequence encoding an N terminal methionine or heterologous signal peptide which is not included when calculating percent homology. Fragments can further comprise coding sequences for an N terminal methionine and/or a signal peptide such as an immunoglobulin signal peptide, for example an IgE or IgG signal peptide. The coding sequence encoding the N terminal methionine and/or signal peptide may be linked to the fragment.

Fragments of SEQ ID NO:9 may comprise 90 or more nucleotides. In some embodiments, fragments of SEQ ID NO:9 may comprise 180 or more nucleotides; in some embodiments, 270 or more nucleotides; in some embodiments 360 or more nucleotides; in some embodiments, 450 or more nucleotides; in some embodiments 540 or more nucleotides; in some embodiments, 630 or more nucleotides; in some embodiments, 720 or more nucleotides; and in some embodiments, 770 or more nucleotides. In some embodiments, fragments of SEQ ID NO:9 such as those set forth herein may further comprise coding sequences for the IgE leader sequences. In some embodiments, fragments of SEQ ID NO:9 do not comprise coding sequences for the IgE leader sequences. Fragments of SEQ ID NO:9 may comprise fewer than 180 nucleotides, in some embodiments fewer than 270 nucleotides, in some embodiments fewer than 360 nucleotides, in some embodiments fewer than 450 nucleotides, in some embodiments fewer than 540 nucleotides, in some embodiments fewer than 630 nucleotides, in some embodiments fewer than 690 nucleotides, in some embodiments fewer than 760 nucleotides, and in some embodiments fewer than 780 nucleotides.

Fragments of SEQ ID NO:10 may comprise 30 or more amino acids. In some embodiments, fragments of SEQ ID NO:10 may comprise 60 or more amino acids; in some embodiments, 90 or more amino acids; in some embodiments, 120 or more amino acids; in some embodiments; 150 or more amino acids; in some embodiments 180 or more amino acids; in some embodiments, 210 or more amino acids; and in some embodiments, 240 or more amino acids. Fragments may comprise fewer than 90 amino acids, in some embodiments fewer than 120 amino acids, in some embodiments fewer than 150 amino acids, in some embodiments fewer than 180 amino acids, in some embodiments fewer than 210 amino acids, and in some embodiments fewer than 240 amino acids.

All fragments of SEQ ID NO:13 comprise coding sequences encoding HPV sequences, i.e. the fragments of SEQ ID NO:13 must comprise sequences in addition to those encoding the IgE leader peptide. In some embodiments, fragments of SEQ ID NO:13 comprise 90 or more nucleotides. In some embodiments, fragments of SEQ ID NO:13 may comprise 180 or more nucleotides; in some embodiments, 270 or more nucleotides; in some embodiments 360 or more nucleotides; in some embodiments, 450 or more nucleotides; in some embodiments 540 or more nucleotides; in some embodiments, 630 or more nucleotides; in some embodiments, 720 or more nucleotides; in some embodiments, 810 or more nucleotides; and in some embodiments, 830 or more nucleotides. Fragments of SEQ ID NO:13 may comprise fewer than 180 nucleotides, in some embodiments fewer than 270 nucleotides, in some embodiments fewer than 360 nucleotides, in some embodiments fewer than 450 nucleotides, in some embodiments fewer than 540 nucleotides, in some embodiments fewer than 630 nucleotides, in some embodiments fewer than 690 nucleotides, in some embodiments fewer than 720 nucleotides, in some embodiments fewer than 780 nucleotides, and in some embodiments fewer than 840 nucleotides.

Fragments of SEQ ID NO:14 may comprise 30 or more amino acids including HPV sequences. In some embodiments, fragments of SEQ ID NO:14 may comprise 60 or more amino acids including HPV sequences; in some embodiments, 90 or more amino acids including HPV sequences; in some embodiments, 120 or more amino acids including HPV sequences; in some embodiments; 150 or more amino acids including HPV sequences; in some embodiments 180 or more amino acids including HPV sequences; in some embodiments, 210 or more amino acids including HPV sequences; in some embodiments, 240 or more amino acids including HPV sequences; and in some embodiments, 270 or more amino acids including HPV sequences. Fragments may comprise fewer than 90 amino acids including HPV sequences, in some embodiments fewer than 120 amino acids including HPV sequences, in some embodiments fewer than 150 amino acids including HPV sequences, in some embodiments fewer than 180 amino acids including HPV sequences, in some embodiments fewer than 210 amino acids including HPV sequences, in some embodiments fewer than 240 amino acids including HPV sequences, and in some embodiments fewer than 270 amino acids including HPV sequences.

In one embodiment, the HPV16 E6-E7 immunogen, HPV18 E6-E7 immunogen; or nucleic acid molecule encoding the HPV16 E6-E7 immunogen or HPV16 E6-E7 immunogen is administered in combination with IL-12. In one embodiment, IL-12 is encoded from a synthetic DNA plasmid.

Methods of treating or preventing anal high-grade squamous intraepithelial lesion in a subject by inducing an immune response in an individual against HPV comprising administering to said individual a composition comprising a nucleic acid sequences provided herein. In some embodiments, the methods also include introducing the nucleic acid sequences into the individual by electroporation.

In some aspects, there are methods of treating or preventing anal high-grade squamous intraepithelial lesion in a subject by inducing an immune response in an individual against HPV comprising administering to said individual a composition comprising a amino acid sequence provided herein. In some embodiments, the methods also include introducing the amino acid sequences into the individual by electroporation.

Improved vaccines comprise proteins and genetic constructs that encode proteins with epitopes that make them particularly effective as immunogens against which anti-HPV immune responses can be induced. Accordingly, vaccines can be provided to induce a therapeutic or prophylactic immune response. In some embodiments, the means to deliver the immunogen is a DNA vaccine, a recombinant vaccine, a protein subunit vaccine, a composition comprising the immunogen, an attenuated vaccine or a killed vaccine. In some embodiments, the vaccine comprises a combination selected from the groups consisting of: one or more DNA vaccines, one or more recombinant vaccines, one or more protein subunit vaccines, one or more compositions comprising the immunogen, one or more attenuated vaccines and one or more killed vaccines.

Aspects of the invention provide methods of delivering the coding sequences of the protein on nucleic acid molecule such as plasmid, as part of recombinant vaccines and as part of attenuated vaccines, as isolated proteins or proteins part of a vector.

According to some aspects of the present invention, compositions and methods are provided which prophylactically and/or therapeutically immunize an individual.

DNA vaccines are described in U.S. Pat. Nos. 5,593,972, 5,739,118, 5,817,637, 5,830,876, 5,962,428, 5,981,505, 5,580,859, 5,703,055, 5,676,594, and the priority applications cited therein, which are each incorporated herein by reference. In addition to the delivery protocols described in those applications, alternative methods of delivering DNA are described in U.S. Pat. Nos. 4,945,050 and 5,036,006, which are both incorporated herein by reference.

The present invention relates to improved attenuated live vaccines, improved killed vaccines and improved vaccines that use recombinant vectors to deliver foreign genes that encode antigens and well as subunit and glycoprotein vaccines. Examples of attenuated live vaccines, those using recombinant vectors to deliver foreign antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference.

When taken up by a cell, the genetic construct(s) may remain present in the cell as a. functioning extrachromosomal molecule and/or integrate into the cell's chromosomal DNA. DNA may be introduced into cells where it remains as separate genetic material in the form of a plasmid or plasmids. Alternatively, linear DNA that can integrate into the chromosome may be introduced into the cell. When introducing DNA into the cell, reagents that promote DNA integration into chromosomes may be added. DNA sequences that are useful to promote integration may also be included in the DNA molecule. Alternatively, RNA may be administered to the cell. It is also contemplated to provide the genetic construct as a linear minichromosome including a centromere, telomeres and an origin of replication. Gene constructs may remain part of the genetic material in attenuated live microorganisms or recombinant microbial vectors which live in cells. Gene constructs may be part of genomes of recombinant viral vaccines where the genetic material either integrates into the chromosome of the cell or remains extrachromosomal. Genetic constructs include regulatory elements necessary for gene expression of a nucleic acid molecule. The elements include: a promoter, an initiation codon, a stop codon, and a polyadenylation signal. In addition, enhancers are often required for gene expression of the sequence that encodes the target protein or the immunomodulating protein. It is necessary that these elements be operable linked to the sequence that encodes the desired proteins and that the regulatory elements are operably in the individual to whom they are administered.

Initiation codons and stop codon are generally considered to be part of a nucleotide sequence that encodes the desired protein. However, it is necessary that these elements are functional in the individual to whom the gene construct is administered. The initiation and termination codons must be in frame with the coding sequence.

Promoters and polyadenylation signals used must be functional within the cells of the individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (MV) such as the BIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein.

Examples of polyadenylation signals useful to practice the present invention, especially in the production of a genetic vaccine for humans, include but are not limited to SV40 polyadenylation signals and LTR polyadenylation signals. In particular, the SV40 polyadenylation signal that is in pCEP4 plasmid (Invitrogen, San Diego Calif.), referred to as the SV40 polyadenylation signal, is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with mammalian origin of replication in order to maintain the construct extrachromosomally and produce multiple copies of the construct in the cell. Plasmids pVAX1, pCEP4 and pREP4 from Invitrogen (San Diego, Calif.) contain the Epstein Barr virus origin of replication and nuclear antigen EBNA-1 coding region which produces high copy episomal replication without integration.

In some preferred embodiments related to immunization applications, nucleic acid molecule(s) are delivered which include nucleotide sequences that encode protein of the invention and, additionally, genes for proteins which further enhance the immune response against such target proteins. Examples of such genes are those which encode other cytokines and lymphokines such as alpha-interferon, gamma-interferon, platelet derived growth factor (PDGF), TNFα, TNFβ, GM-CSF, epidermal growth factor (EGF), IL-1, IL-2, IL-4, IL-5, IL-6, IL-10, IL-12, IL-18, MHC, CD80, CD86 and IL-15 including IL-15 having the signal sequence deleted and optionally including the signal peptide from IgE. Other genes which may be useful include those encoding: MCP-1, MIP-1α, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

An additional element may be added which serves as a target for cell destruction if it is desirable to eliminate cells receiving the genetic construct for any reason. A herpes thymidine kinase (tk) gene in an expressible form can be included in the genetic construct. The drug gangcyclovir can be administered to the individual and that drug will cause the selective killing of any cell producing tk, thus, providing the means for the selective destruction of cells with the genetic construct.

In order to maximize protein production, regulatory sequences may be selected which are well suited for gene expression in the cells the construct is administered into. Moreover, codons may be selected which are most efficiently transcribed in the cell. One having ordinary skill in the art can produce DNA constructs that are functional in the cells.

In some embodiments, gene constructs may be provided in which the coding sequences for the proteins described herein are linked to IgE signal peptide. In some embodiments, proteins described herein are linked to IgE signal peptide.

In some embodiments for which protein is used, for example, one having ordinary skill in the art can, using well known techniques, produce and isolate proteins of the invention using well known techniques. In some embodiments for which protein is used, for example, one having ordinary skill in the art can, using well known techniques, inserts DNA molecules that encode a protein of the invention into a commercially available expression vector for use in well known expression systems. For example, the commercially available plasmid pSE420 (Invitrogen, San Diego, Calif.) may be used for production of protein in E. coli. The commercially available plasmid pYES2 (Invitrogen, San Diego, Calif.) may, for example, be used for production in S. cerevisiae strains of yeast. The commercially available MAXBAC™ complete baculovirus expression system (Invitrogen, San Diego, Calif.) may, for example, be used for production in insect cells. The commercially available plasmid pcDNA I or pcDNA3 (Invitrogen, San Diego, Calif.) may, for example, be used for production in mammalian cells such as Chinese Hamster Ovary cells. One having ordinary skill in the art can use these commercial expression vectors and systems or others to produce protein by routine techniques and readily available starting materials. (See e.g., Sambrook et al., Molecular Cloning a Laboratory Manual, Second Ed. Cold Spring Harbor Press (1989) which is incorporated herein by reference.) Thus, the desired proteins can be prepared in both prokaryotic and eukaryotic systems, resulting in a spectrum of processed forms of the protein.

One having ordinary skill in the art may use other commercially available expression vectors and systems or produce vectors using well known methods and readily available starting materials. Expression systems containing the requisite control sequences, such as promoters and polyadenylation signals, and preferably enhancers are readily available and known in the art for a variety of hosts. See e.g., Sambrook et al., Molecular Cloning a Laboratory Manual, Second Ed. Cold Spring Harbor Press (1989). Genetic constructs include the protein coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes protein of the invention from readily available starting materials. The expression vector including the DNA that encodes the protein is used to transform the compatible host which is then cultured and maintained under conditions wherein expression of the foreign DNA takes place.

The protein produced is recovered from the culture, either by lysing the cells or from the culture medium as appropriate and known to those in the art. One having ordinary skill in the art can, using well known techniques, isolate protein that is produced using such expression systems. The methods of purifying protein from natural sources using antibodies which specifically bind to a specific protein as described above may be equally applied to purifying protein produced by recombinant DNA methodology.

In addition to producing proteins by recombinant techniques, automated peptide synthesizers may also be employed to produce isolated, essentially pure protein. Such techniques are well known to those having ordinary skill in the art and are useful if derivatives which have substitutions not provided for in DNA-encoded protein production.

The nucleic acid molecules may be delivered using any of several well known technologies including DNA injection (also referred to as DNA vaccination), recombinant vectors such as recombinant adenovirus, recombinant adenovirus associated virus and recombinant vaccinia.

Routes of administration include, but are not limited to, intramuscular, intransally, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. Preferred routes of administration include intramuscular, intraperitoneal, intradermal and subcutaneous injection. Genetic constructs may be administered by means including, but not limited to, electroporation methods and devices, traditional syringes, needleless injection devices, or “microprojectile bombardment gone guns”.

Examples of electroporation devices and electroporation methods preferred for facilitating delivery of the DNA vaccines, include those described in U.S. Pat. No. 7,245,963 by Draghia-Akli, et al., U.S. Patent Pub. 2005/0052630 submitted by Smith, et al., the contents of which are hereby incorporated by reference in their entirety. Also preferred, are electroporation devices and electroporation methods for facilitating delivery of the DNA vaccines provided in co-pending and co-owned U.S. patent application Ser. No. 11/874,072, filed Oct. 17, 2007, which claims the benefit under 35 USC 119(e) to U.S. Provisional Application Ser. No. 60/852,149, filed Oct. 17, 2006, and 60/978,982, filed Oct. 10, 2007, all of which are hereby incorporated in their entirety.

The following is an example of an embodiment using electroporation technology, and is discussed in more detail in the patent references discussed above: electroporation devices can be configured to deliver to a desired tissue of a mammal a pulse of energy producing a constant current similar to a preset current input by a user. The electroporation device comprises an electroporation component and an electrode assembly or handle assembly. The electroporation component can include and incorporate one or more of the various elements of the electroporation devices, including: controller, current waveform generator, impedance tester, waveform logger, input element, status reporting element, communication port, memory component, power source, and power switch. The electroporation component can function as one element of the electroporation devices, and the other elements are separate elements (or components) in communication with the electroporation component. In some embodiments, the electroporation component can function as more than one element of the electroporation devices, which can be in communication with still other elements of the electroporation devices separate from the electroporation component. The use of electroporation technology to deliver the improved HPV vaccine is not limited by the elements of the electroporation devices existing as parts of one electromechanical or mechanical device, as the elements can function as one device or as separate elements in communication with one another. The electroporation component is capable of delivering the pulse of energy that produces the constant current in the desired tissue, and includes a feedback mechanism. The electrode assembly includes an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism can receive the measured impedance and can adjust the pulse of energy delivered by the electroporation component to maintain the constant current.

In some embodiments, the plurality of electrodes can deliver the pulse of energy in a decentralized pattern. In some embodiments, the plurality of electrodes can deliver the pulse of energy in the decentralized pattern through the control of the electrodes under a programmed sequence, and the programmed sequence is input by a user to the electroporation component. In some embodiments, the programmed sequence comprises a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

In some embodiments, the feedback mechanism is performed by either hardware or software. Preferably, the feedback mechanism is performed by an analog closed-loop circuit. Preferably, this feedback occurs every 50 μs, 20 μs, 10 μs or 1 μs, but is preferably a real-time feedback or instantaneous (i.e., substantially instantaneous as determined by available techniques for determining response time). In some embodiments, the neutral electrode measures the impedance in the desired tissue and communicates the impedance to the feedback mechanism, and the feedback mechanism responds to the impedance and adjusts the pulse of energy to maintain the constant current at a value similar to the preset current. In some embodiments, the feedback mechanism maintains the constant current continuously and instantaneously during the delivery of the pulse of energy.

In some embodiments, the nucleic acid molecule is delivered to the cells in conjunction with administration of a polynucleotide function enhancer or a genetic vaccine facilitator agent. Polynucleotide function enhancers are described in U.S. Pat. Nos. 5,593,972, 5,962,428 and International Application Serial Number PCT/US94/00899 filed Jan. 26, 1994, which are each incorporated herein by reference. Genetic vaccine facilitator agents are described in U.S. Pat. No. 021,579 filed Apr. 1, 1994, which is incorporated herein by reference. The co-agents that are administered in conjunction with nucleic acid molecules may be administered as a mixture with the nucleic acid molecule or administered separately simultaneously, before or after administration of nucleic acid molecules. In addition, other agents which may function transfecting agents and/or replicating agents and/or inflammatory agents and which may be co-administered with a GVF include growth factors, cytokines and lymphokines such as a-interferon, gamma-interferon, GM-CSF, platelet derived growth factor (PDGF), TNF, epidermal growth factor (EGF), IL-1, IL-2, IL-4, IL-6, IL-10, IL-12 and IL-15 as well as fibroblast growth factor, surface active agents such as immune-stimulating complexes (ISCOMS), Freunds incomplete adjuvant, LPS analog including monophosphoryl Lipid A (WL), muramyl peptides, quinone analogs and vesicles such as squalene and squalene, and hyaluronic acid may also be used administered in conjunction with the genetic construct In some embodiments, an immunomodulating protein may be used as a GVF. In some embodiments, the nucleic acid molecule is provided in association with PLG to enhance delivery/uptake.

The pharmaceutical compositions according to the present invention comprise about 1 nanogram to about 2000 micrograms of DNA. In some preferred embodiments, pharmaceutical compositions according to the present invention comprise about 5 nanogram to about 1000 micrograms of DNA. In some preferred embodiments, the pharmaceutical compositions contain about 10 nanograms to about 800 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms of DNA. In some preferred embodiments, the pharmaceutical compositions contain about 100 to about 200 microgram DNA.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

Provided herein are methods of treating human papillomavirus (HPV) type 16- or HPV type 18-related anal or anal/peri-anal high grade intraepithelial lesion (HSIL) in a subject in need thereof, said method comprising, consisting of, or consisting essentially of: administering a therapeutically effective amount of a pharmaceutical composition in accordance with the invention to the subject. According to some embodiments, the pharmaceutical composition is VGX-3100 or a biosimilar thereof.

Also provided herein is VGX-3100 for use in a method of treating human papillomavirus (HPV) type 16- or HPV type 18-related anal or anal/peri-anal high grade intraepithelial lesion (HSIL) in a subject in need thereof, said method comprising, consisting of, or consisting essentially of: administering a therapeutically effective amount of a pharmaceutical composition in accordance with the invention to the subject. According to some embodiments, the pharmaceutical composition is VGX-3100 or a biosimilar thereof.

Also provided herein are uses of VGX-3100 in the manufacture of a medicament for treating human papillomavirus (HPV) type 16- or HPV type 18-related anal or anal/peri-anal high grade intraepithelial lesion (HSIL), said method comprising, consisting of, or consisting essentially of: administering a therapeutically effective amount of a pharmaceutical composition in accordance with the invention to the subject. According to some embodiments, the pharmaceutical composition is VGX-3100 or a biosimilar thereof.

Also provided herein are methods of improving histopathologic regression of anal or anal/peri-anal HSIL in a subject in need thereof comprising, consisting of, or consisting essentially of administering a therapeutically effective amount of a pharmaceutical composition in accordance with the invention to the subject. According to some embodiments, the pharmaceutical composition is VGX-3100 or a biosimilar thereof. In certain embodiments, the improvement in histopathologic regression of anal or anal/peri-anal HSIL is relative to no treatment of a subject or a population of subjects. In certain embodiments, the improvement in histopathologic regression of anal or anal/peri-anal HSIL is relative to treatment of a subject of population of subjects with the standard of care.

Also provided herein are methods of improving virologic clearance of HPV-16 and/or HPV-18 in a subject having anal or anal/peri-anal HSIL comprising, consisting of, or consisting essentially of administering a therapeutically effective amount of a pharmaceutical composition in accordance with the invention to the subject. According to some embodiments, the pharmaceutical composition is VGX-3100 or a biosimilar thereof.

Also provided herein are methods of achieving partial or complete histopathologic regression to normal or histopathologic nonprogression of anal or anal/peri-anal HSIL in a subject in a subject who has HPV type 16- or HPV type 18-related anal or peri-anal HSIL comprising, consisting of, or consisting essentially of administering a therapeutically effective amount of a pharmaceutical composition in accordance with the invention to the subject. According to some embodiments, the pharmaceutical composition is VGX-3100 or a biosimilar thereof. In certain embodiments, the achievement of partial or complete histopathologic regression to normal or histopathologic nonprogression of anal or anal/peri-anal HSIL is relative to administration of a placebo to a subject or a population of subjects. In certain embodiments, the achievement of partial or complete histopathologic regression to normal or histopathologic nonprogression of anal or anal/peri-anal HSIL is relative to no treatment of a subject or a population of subjects. In certain embodiments, the achievement of partial or complete histopathologic regression or histopathologic nonprogression of anal or anal/peri-anal HSIL is relative to treatment of a subject of population of subjects with the standard of care.

Also provided herein are methods of improving clearance of HPV-16 and/or HPV-18 infection from nonanal anatomic locations in a subject who has HPV type 16- or HPV type 18-related anal or peri-anal HSIL comprising, consisting of, or consisting essentially of administering a therapeutically effective amount of a pharmaceutical composition in accordance with the invention to the subject. According to some embodiments, the pharmaceutical composition is VGX-3100 or a biosimilar thereof.

In some embodiments, the VGX-3100 is administered to the subject by intramuscular injection followed by electroporation. In certain embodiments, the VGX-3100 is administered to the subject at a dose of 6 mg. In further embodiments, the VGX-3100 is administered to the subject three times over the course of 12 weeks. In further embodiments, the VGX-3100 is administered to the subject four times over the course of 40 weeks. In still further embodiments, the VGX-3100 is formulated at a concentration of 6 mg/ml in 150 mM sodium chloride and 15 mM sodium citrate.

In certain embodiments, administration of VGX-3100 results in virologic clearance of HPV-16 and/or HPV-18 and histopathologic regression of anal HSIL. In further embodiments, administration of VGX-3100 results in histopathologic regression of anal HSIL. In still further embodiments, administration of VGX-3100 results in virologic clearance of HPV-16 and/or HPV-18. In certain embodiments, administration of VGX-3100 results in complete histopathologic regression of anal HSIL to normal. In further embodiments, administration of VGX-3100 results in complete histopathologic regression of anal HSIL to normal and virologic clearance of HPV-16 and/or HPV-18. In still further embodiments, administration of VGX-3100 results in histopathologic nonprogression. In certain embodiments, administration of VGX-3100 results in clearance of HPV-16 and/or HPV-18 infection from nonanal anatomic locations. In further embodiments, administration of VGX-3100 results in improved humoral and cellular immune response to VGX-3100 following a third administration of VGX-3100 and at 36 weeks following administration of VGX-3100 as assessed relative to baseline.

In further embodiments, administration of VGX-3100 results in improved humoral and cellular immune response to VGX-3100 following a fourth administration of VGX-3100 as assessed relative to baseline.

In certain embodiments, the result of VGX-3100 administration is evaluated at 36 weeks following administration of VGX-3100.

According to some embodiments of the invention, methods of inducing immune responses are provided. The vaccine may be a protein based, live attenuated vaccine, a cell vaccine, a recombinant vaccine or a nucleic acid or DNA vaccine. In some embodiments, methods of inducing an immune response in individuals against an immunogen, including methods of inducing mucosal immune responses, comprise administering to the individual one or more of CTACK protein, TECK protein, MEC protein and functional fragments thereof or expressible coding sequences thereof in combination with an isolated nucleic acid molecule that encodes protein of the invention and/or a recombinant vaccine that encodes protein of the invention and/or a subunit vaccine that protein of the invention and/or a live attenuated vaccine and/or a killed vaccine. The one or more of CTACK protein, TECK protein, MEC protein and functional fragments thereof may be administered prior to, simultaneously with or after administration of the isolated nucleic acid molecule that encodes an immunogen; and/or recombinant vaccine that encodes an immunogen and/or subunit vaccine that comprises an immunogen and/or live attenuated vaccine and/or killed vaccine. In some embodiments, an isolated nucleic acid molecule that encodes one or more proteins of selected from the group consisting of: CTACK, TECK, MEC and functional fragments thereof is administered to the individual.

The present invention is further illustrated in the following Example. It should be understood that this Example, while indicating embodiments of the invention, is given by way of illustration only. From the above discussion and this Example, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

Each of the U.S. Patents, U.S. Applications, and references cited throughout this disclosure are hereby incorporated in their entirety by reference.

### Illustrative Embodiments

Embodiment 1. A method for treating or preventing anal high-grade squamous intraepithelial lesion in an individual comprising administering to the individual a composition comprising at least one nucleic acid molecule encoding at least one selected from the group consisting of: an HPV16 antigen and an HPV18 antigen.

Embodiment 2. The method of Embodiment 1, wherein the HPV16 antigen is an HPV16 E6-E7 fusion antigen.

Embodiment 3. The method of Embodiment 1 or 2, wherein the HPV16 antigen comprises the amino acid sequence of SEQ ID NO: 5 and the amino acid sequence of SEQ ID NO: 6.

Embodiment 4. The method of any preceding Embodiment, wherein the HPV16 antigen is encoded by a nucleic acid molecule comprising the nucleotide sequence of SEQ ID NO: 17 and the nucleotide sequence of SEQ ID NO: 19.

Embodiment 5. The method of any preceding Embodiment, wherein the HPV18 antigen is an HPV18 E6-E7 fusion antigen.

Embodiment 6. The method of any preceding Embodiment, wherein the HPV18 antigen comprises the amino acid sequence of SEQ ID NO: 21 and the amino acid sequence of SEQ ID NO: 22.

Embodiment 7. The method of any preceding Embodiment, wherein the composition comprises a nucleotide sequence encoding an HPV16 antigen and a nucleotide sequence encoding an HPV18 antigen.

Embodiment 8. The method of any preceding Embodiment, wherein the nucleic acid molecule comprises one or more nucleotide sequences selected from the group consisting of:

a nucleotide sequence that encodes SEQ ID NO:2;

a nucleotide sequence that is at least 95% homologous to a nucleotide sequence that encodes SEQ ID NO:2;

a fragment of a nucleotide sequence that encodes SEQ ID NO:2;

a nucleotide sequence that is at least 95% homologous to a fragment of a nucleotide sequence that encodes SEQ ID NO:2.

Embodiment 9. The method of Embodiment 8, wherein the nucleic acid molecule comprises a nucleotide sequence that is at least 98% homologous to a nucleotide sequence that encodes SEQ ID NO:2.

Embodiment 10. The method of Embodiment 8, wherein the nucleic acid molecule comprises a nucleotide sequence that is at least 99% homologous to a nucleotide sequence that encodes SEQ ID NO:2.

Embodiment 11. The method of any preceding Embodiment, wherein the nucleic acid molecule comprises one or more nucleotide sequences selected from the group consisting of:

a nucleotide sequence comprising nucleotides 19-795 of SEQ ID NO:1;

a nucleotide sequence comprising nucleotides 1-795 of SEQ ID NO:1;

a nucleotide sequence comprising SEQ ID NO:1;

a nucleotide sequence that is at least 95% homologous SEQ ID NO:1;

a fragment of SEQ ID NO:1;

a nucleotide sequence that is at least 95% homologous to a fragment of SEQ ID NO:1.

Embodiment 12. The method of Embodiment 11, wherein the nucleic acid molecule comprises a nucleotide sequence that is at least 98% homologous to SEQ ID NO:1.

Embodiment 13. The method of Embodiment 11, wherein the nucleic acid molecule comprises a nucleotide sequence that is at least 99% homologous to SEQ ID NO:1.

Embodiment 14. The method of any preceding Embodiment, wherein the nucleic acid molecule comprises the nucleotide sequence of SEQ ID NO: 23 and the nucleotide sequence of SEQ ID NO: 24.

Embodiment 15. The method of any preceding Embodiment, wherein the nucleic acid molecule comprises one or more nucleotide sequences selected from the group consisting of:

a nucleotide sequence that encodes SEQ ID NO:10;

a nucleotide sequence that is at least 95% homologous to a nucleotide sequence that encodes SEQ ID NO:10;

a fragment of a nucleotide sequence that encodes SEQ ID NO:10;

a nucleotide sequence that is at least 95% homologous to a fragment of a nucleotide sequence that encodes SEQ ID NO:10.

Embodiment 16. The method of Embodiment 15, wherein the nucleic acid molecule comprises a nucleotide sequence that is at least 98% homologous to a nucleotide sequence that encodes SEQ ID NO:10.

Embodiment 17. The method of Embodiment 15, wherein the nucleic acid molecule comprises a nucleotide sequence that is at least 99% homologous to a nucleotide sequence that encodes SEQ ID NO:10.

Embodiment 18. The method of any one of Embodiments 1-7 or 14-17, wherein the nucleotide acid molecule encoding the HPV16 E6-E7 fusion antigen further comprises a nucleotide sequence encoding a leader sequence, wherein the nucleotide sequence encoding the HPV18 E6-E7 fusion antigen further comprises a nucleotide sequence encoding a leader sequence, or wherein the nucleotide sequence encoding the HPV16 E6-E7 fusion antigen further comprises a nucleotide sequence encoding a leader sequence and the nucleotide sequence encoding the HPV18 E6-E7 fusion antigen further comprises a nucleotide sequence encoding a leader sequence.

Embodiment 19. The method of any preceding Embodiment, wherein the nucleic acid molecule comprises one or more nucleotide sequences selected from the group consisting of:

a nucleotide sequence comprising nucleotides 1-780 of SEQ ID NO:9;

a nucleotide sequence comprising SEQ ID NO:9;

a nucleotide sequence that is at least 95% homologous SEQ ID NO:9;

a fragment of SEQ ID NO:9;

a nucleotide sequence that is at least 95% homologous to a fragment of SEQ ID NO:9.

Embodiment 20. The method of Embodiment 19, wherein the nucleic acid molecule comprises a nucleotide sequence that is at least 98% homologous to SEQ ID NO:9.

Embodiment 21. The method of Embodiment 19, wherein the nucleic acid molecule comprises a nucleotide sequence that is at least 99% homologous to SEQ ID NO:9.

Embodiment 22. The method of any preceding Embodiment, wherein the at least one nucleic acid molecule comprises at least one plasmid.

Embodiment 23. The method of any preceding Embodiment, wherein the composition is a pharmaceutical composition.

Embodiment 24. The method of any preceding Embodiment, further comprising administering to the individual a composition comprising an adjuvant.

Embodiment 25. The method of any preceding Embodiment, comprising administering to the individual a nucleic acid molecule comprising a nucleotide sequence encoding an HPV16 E6-E7 fusion antigen and a nucleic acid molecule comprising a nucleotide sequence encoding an HPV18 E6-E7 fusion antigen;

wherein the nucleotide sequence encoding the HPV16 E6-E7 fusion antigen is selected from the group consisting of: a nucleotide sequence that encodes SEQ ID NO:2; a nucleotide sequence that is at least 95% homologous to a nucleotide sequence that encodes SEQ ID NO:2; a fragment of a nucleotide sequence that encodes SEQ ID NO:2; and a nucleotide sequence that is at least 95% homologous to a fragment of a nucleotide sequence that encodes SEQ ID NO:2; and

wherein the nucleotide sequence encoding the HPV18 E6-E7 fusion antigen is selected from the group consisting of: a nucleotide sequence that encodes SEQ ID NO:10;

a nucleotide sequence that is at least 95% homologous to a nucleotide sequence that encodes SEQ ID NO:10; a fragment of a nucleotide sequence that encodes SEQ ID NO:10; and a nucleotide sequence that is at least 95% homologous to a fragment of a nucleotide sequence that encodes SEQ ID NO:10.

Embodiment 26. The method of Embodiment 25, wherein one nucleic acid molecule comprises the nucleotide sequence encoding an HPV16 E6-E7 fusion antigen and the nucleotide sequence encoding an HPV18 E6-E7 fusion antigen.

Embodiment 27. The method of any preceding Embodiment, wherein administering said nucleic acid molecule to the individual comprises electroporation.

Embodiment 28. The method of any preceding Embodiment, wherein the composition is administered intramuscularly.

Embodiment 29. The method of any preceding Embodiment, wherein the composition comprises VGX-3100 or a biosimilar thereof.

Embodiment 30. The method of any preceding Embodiment, wherein the composition is administered in three or four doses.

Embodiment 31. The method of Embodiment 30, wherein the second dose of the composition is administered about four weeks after the first dose.

Embodiment 32. The method of Embodiment 30, wherein the third dose is administered about twelve weeks after the first dose.

Embodiment 33. The method of Embodiment 30, wherein the fourth dose is administered about 40 weeks after the first dose.

Embodiment 34. The method of any preceding Embodiment, wherein the HSIL is anal or anal/peri-anal HSIL.

Embodiment 35. The method of Embodiment 34, wherein the leader sequence comprises the amino acid sequence of SEQ ID NO: 7.

Embodiment 36. The method of Embodiment 18, wherein the leader sequence is encoded by a nucleic acid molecule comprising the nucleotide sequence of SEQ ID NO: 11.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: VGX-3100 Delivered Intramuscularly (IM) Followed by Electroporation (EP) for the Treatment of HPV-16 and/or HPV-18 Related Anal or Anal/Peri-Anal, High Grade Squamous Intraepithelial Lesion (HSIL) in Individuals Seronegative for Human Immunodeficiency Virus (HIV)-1/2 (ClinicalTrials_Gov Identifier: NCT03499795)

This is a phase 2, open-label efficacy study of VGX-3100 administered by intramuscular (IM) injection followed by electroporation (EP) in adult men and women who are human immunodeficiency virus (HIV)-negative with histologically confirmed anal or anal/peri-anal high-grade squamous intraepithelial lesion (HSIL) associated with human papilloma virus (HPV)-16 and/or HPV-18.

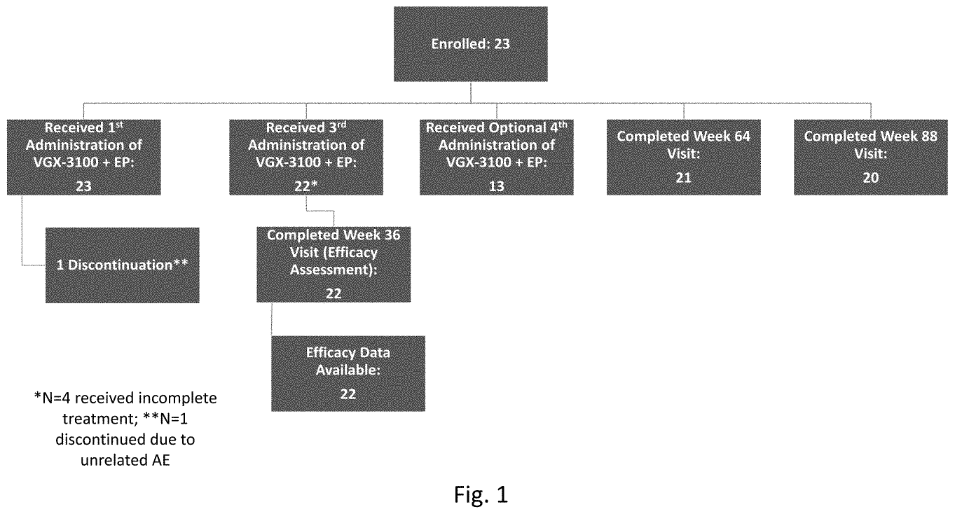

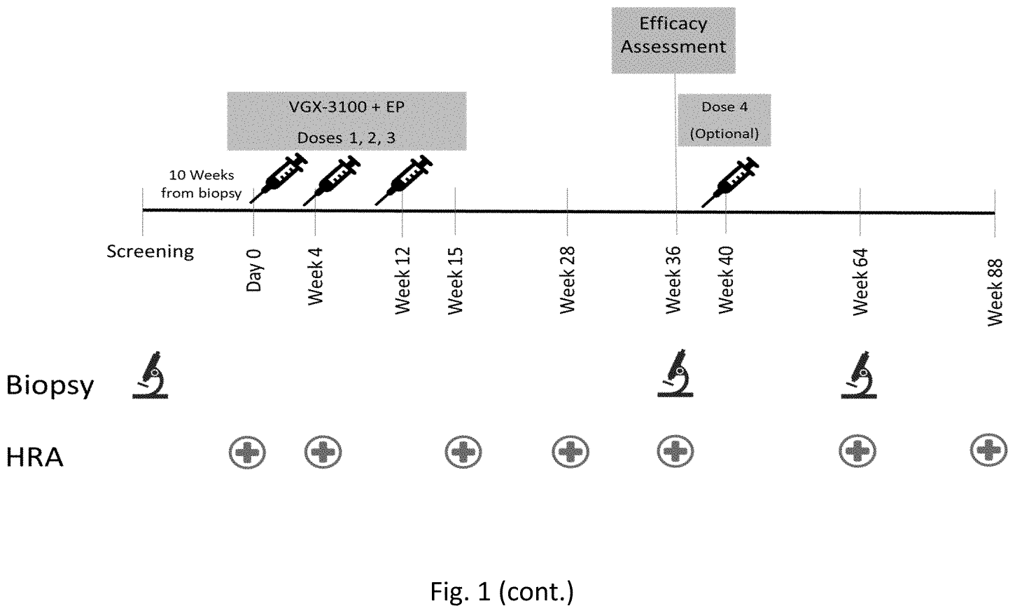

Anal high-grade squamous intraepithelial lesion (HSIL) management remains challenging. VGX-3100 is a DNA-based HPV16/18-specific immunotherapy that encodes for HPV16 & 18 E6/E7 proteins and was assessed in an open-label phase 2 study to treat anal HSIL (FIG. 1).

Inclusion Criteria:

18 years of age or older;

Negative screening test for HIV-1/2 within 30 days of Dose 1;

Confirmed anal or anal/peri-anal HPV-16/18 infection at Screening by polymerase chain reaction (PCR) from HSIL specimen;

At least one anal or anal/peri-anal (AIN2/3 and/or PAIN2/PAIN3) lesion that is histologically-confirmed as HSIL at Screening;

Appropriate candidate for histology collection procedures (i.e. excision or biopsy);

Female subjects must be post-menopausal, surgically sterile or agree to avoid pregnancy by continued abstinence or use of a contraceptive method with failure rate of less than 1% per year from Screening to one month after last dose of study medication (Week 12 or Week 40)

Men who could father a child must agree to use at least one form of birth control during or continued abstinence from heterosexual intercourse prior to the study, for the duration of study participation and one month after last dose of study medication.

Normal Screening electrocardiogram (ECG).

Exclusion Criteria:

Untreated micro invasive or invasive cancer;

Biopsy-proven Vaginal Intraepithelial Neoplasia (VAIN) and not undergoing medical care and/or treatment for VAIN;

Biopsy-proven Vulvar Intraepithelial Neoplasia (VIN) and not undergoing medical care and/or treatment for VIN;

Biopsy-proven Cervical Intraepithelial Neoplasia (CIN) 2/3 and not undergoing medical care and/or treatment for CIN;

Biopsy-proven Penile Intraepithelial Neoplasia (PIN) and not undergoing medical care and/or treatment for PIN;

Anal or anal/peri-anal HSIL that is not accessible for sampling by biopsy instrument;

Intra-anal and/or peri-anal lesion(s) that cannot be fully visualized at Screening;

Inability to have complete and satisfactory high resolution anoscopic exams (HRAs)

Any treatment for anal or anal/peri-anal HSIL (e.g. surgery) within 4 weeks of Screening;

Pregnant, breast feeding or considering becoming pregnant within one month following the last dose of study medication;

Presence of any abnormal clinical laboratory values greater than Grade 1 per Common Toxicity Criteria for Adverse Events (CTCAE) version 4.03 within 45 days prior to Day 0 or less than Grade 1 but deemed clinically significant by the Investigator;

Immunosuppression as a result of underlying illness or treatment;

History of previous therapeutic HPV vaccination;

Receipt of any non-study related non-live vaccine within 2 weeks of any VGX-3100 dose;

Receipt of any non-study related live vaccine (e.g. measles vaccine) within 4 weeks of any VGX-3100 dose;

Significant acute or chronic medical illness that could be negatively impacted by the electroporation treated as deemed by the Investigator;

Current or history of clinically significant, medically unstable disease which, in the judgment of the investigator, would jeopardize the safety of the subject, interfere with study assessments or endpoint evaluation, or otherwise impact the validity of the study results;

Prior major surgery within 4 weeks of Day 0;

Participation in an interventional study with an investigational compound or device;

Any illness or condition that in the opinion of the Investigator may affect the safety of the subject or the evaluation of any study endpoint.

Primary Outcome Measures:

Percentage of Participants with No Histologic Evidence of Anal or Anal/Peri-Anal HSIL and No Evidence of HPV-16/18 at Week 36 [Time Frame: At Week 36]

Secondary Outcome Measures:

Number of Local and Systemic Safety Events During 7 Days Following Each Dose [Time Frame: Days 0-7 (7 days following Day 0 dose), Days 22-29 (7 days following Week 4 dose) and Days 78-85 (7 days following Week 12 dose)]

Number of Adverse Events [Time Frame: From baseline to the Week 88 visit]

Percentage of Participants with No Evidence of Anal or Anal/Peri-Anal HSIL on Histology at the Week 36 visit [Time Frame: At Week 36]

Percentage of Participants with No Evidence of HPV-16/18 from Intra-Anal and/or Peri-Anal Tissue by Type-Specific HPV Testing at the Week 36 Visit [Time Frame: At Week 36]

Percentage of Participants with No Evidence of HPV-16/18 from Intra-Anal Swab by Specific HPV Testing at the Weeks 36, 64, and 88 Visits [Time Frame: At Weeks 36, 64 and 88]

Percentage of Participants with No Evidence of Anal or Anal/Peri-Anal Low-Grade Squamous Intraepithelial Lesion (LSIL) or HSIL on Histology at the Week 36 Visit [Time Frame: At Week 36]

Percentage of Participants with No Progression of Anal or Anal/Peri-Anal HSIL to Carcinoma from Baseline on Histology at the Week 36 Visit [Time Frame: From baseline to Week 36]

Percentage Reduction from Baseline in the Number of Intra-Anal and/or Peri-Anal Lesion(s) as Determined by the Investigator at the Weeks 36, 64 and 88 Visits [Time Frame: From baseline to Weeks 36, 64 and 88]

Percentage Reduction from Baseline in the Size of Peri-Anal Lesion(s) as Determined by the Investigator at the Weeks 36, 64 and 88 Visits [Time Frame: From baseline to Weeks 36, 64 and 88]

Flow Cytometry Response Magnitudes [Time Frame: At baseline and Week 15]

Percentage of Participants with No Histologic Evidence of Anal or Anal/Peri-Anal HSIL or No Evidence of HPV-16/18 at Week 36 [Time Frame: At Week 36]

Investigational Product

Common name: VGX-3100

Chemical name: Circular, double stranded, deoxyribonucleic acid consisting of 3782 base pairs for the pGX3001 plasmid and 3824 base pairs for the pGX3002 plasmid.

Distinguishing name: Eukaryotic expression plasmids containing HPV 16 and 18-E6 & E7-encoding transcription unit controlled by a synthetic, CMV promoter, and elements required for replication and selection in E. coli, namely a pUC origin of replication (pUC Ori) and a kanamycin resistance gene (Kan R).

Detailed description: VGX-3100, HPV therapeutic vaccine is a combination of two plasmids in equal quantities (i.e. the 6 mg dose will deliver 3 mg of each pGX3001 and pGX3002 plasmids): a) pGX3001: p16ConE6E7, a plasmid encoding for a synthetic HPV16 consensus E6 and E7 fusion gene (“consensus HPV 16-6&7”) into a pVAX1 backbone (Invitrogen, Carlsbad, Calif.) under the control of the cytomegalovirus immediate-early (CMV) promoter, and b) pGX3002: p18ConE6E7, a plasmid encoding for a synthetic HPV18 consensus E6 and E7 fusion gene (“consensus HPV 18-6&7”) into a pVAX1 backbone (Invitrogen, Carlsbad, Calif.) under the control of the cytomegalovirus immediate-early (CMV) promoter. VGX-3100 is described in WO2014/165291, which is incorporated herein by reference in its entirety. The nucleic acid and amino acid consensus sequences are provided in Table 1 and Table 2, respectively.

TABLE 1
VGX-3100 consensus sequences (nucleic acid)

| Consensus Fusion Gene | SEQ ID NO |
|---|---|
| Consensus HPV 16-6&7 | 1 |
| Consensus HPV 18-6&7 | 13 |

TABLE 2
VGX-3100 consensus sequences (amino acid)

| Consensus Fusion Gene | SEQ ID NO |
|---|---|
| Consensus HPV 16-6&7 | 2 |
| Consensus HPV 18-6&7 | 14 |

Subjects with HPV16/18 positive anal HSIL received 3 or 4 doses of VGX-3100 intramuscularly followed by CELLECTRA® electroporation. Subjects underwent biopsy of HSILs and histology-based clinical endpoints were assessed at Week 36 after VGX-3100 treatment onset. Endpoints included resolution of anal HSIL and clearance of HPV16/18 in anal tissue (primary), and reduction of lesion number and size (secondary). In addition, samples to demonstrate concordance between anal swabs and resected tissue for HPV at baseline were collected and used the SPF10-LiPA25 (Line probe assay) version 1 system (tissue) and the Roche Cobas® HPV Test (swabs).

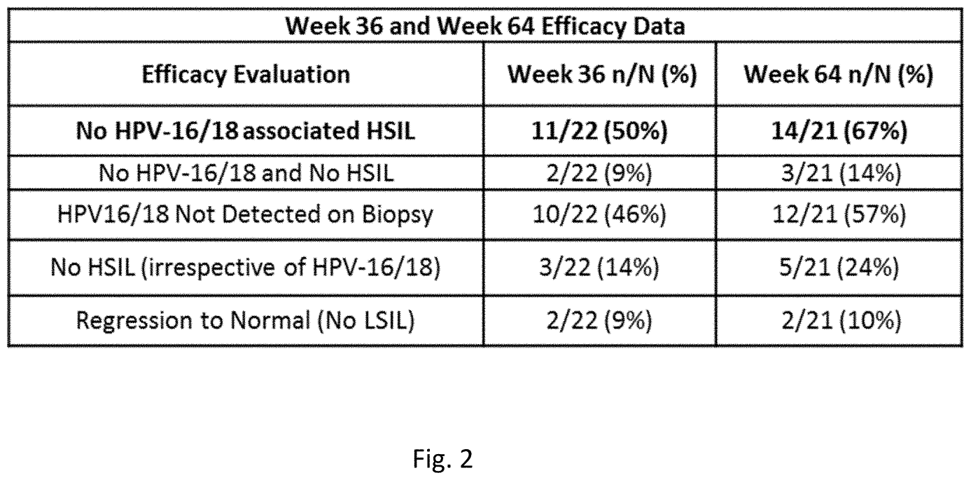

FIG. 2 provides data on the efficacy of the vaccine at weeks 36 and 64.

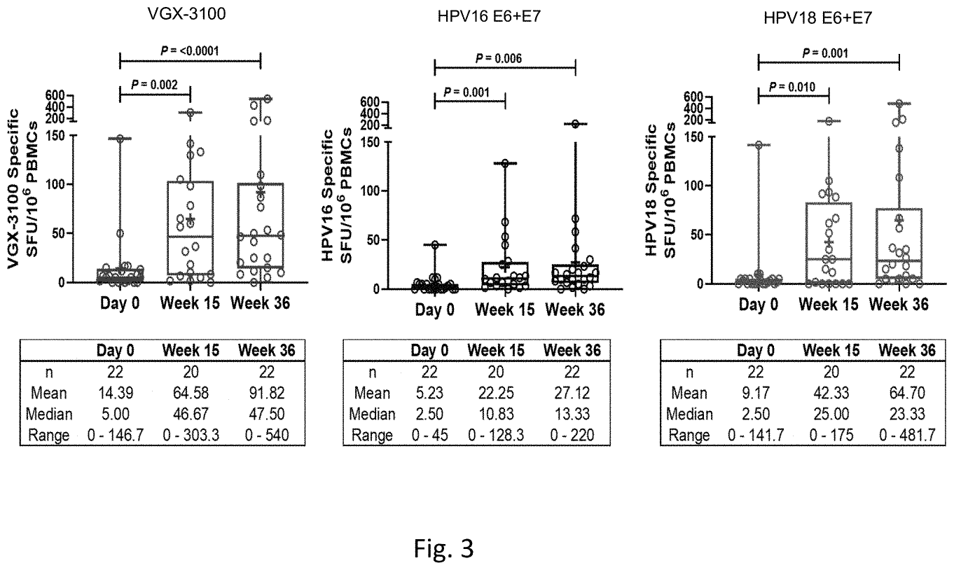

FIG. 3 provides data demonstrating that VGX-3100 induces a cellular immune response to both HPV16 and HPV18 antigens.

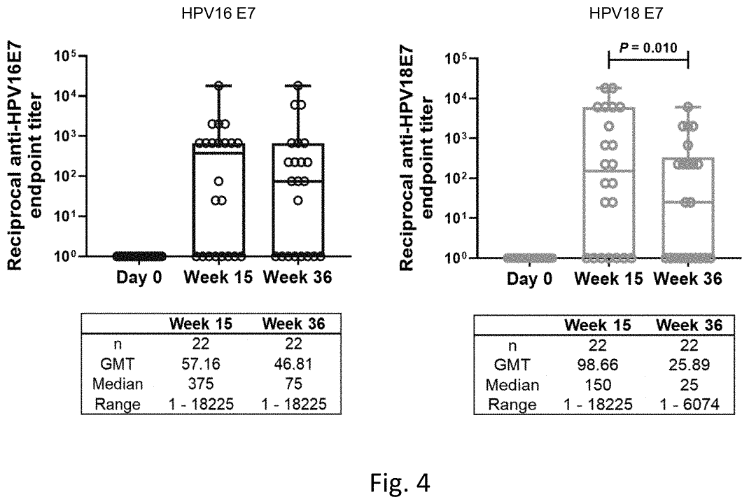

FIG. 4 provides data demonstrating that VGX-3100 induces a humoral immune response to HPV16E7 and HPV18E7 antigens.

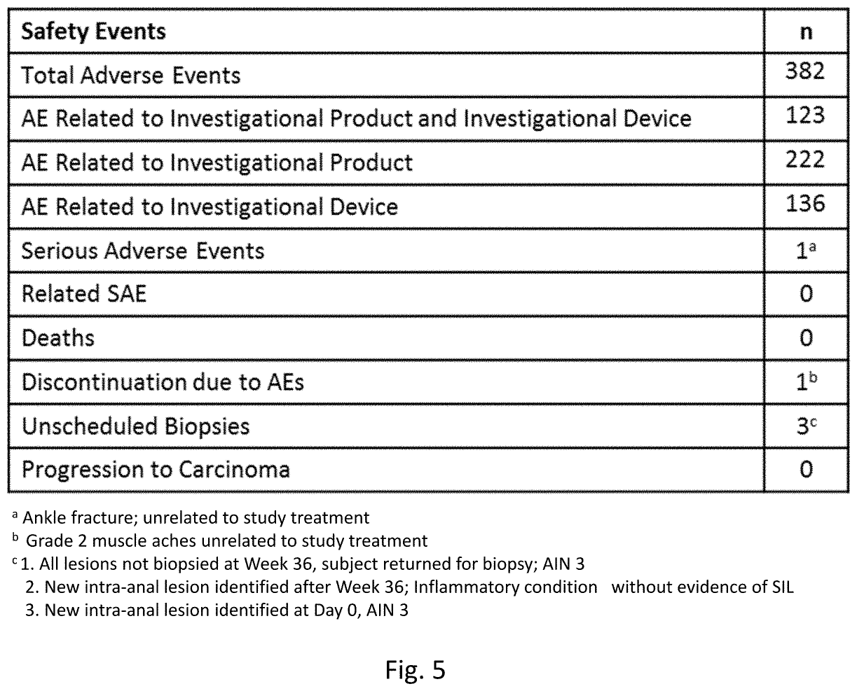

FIG. 5 provides data on the safety of the VGX-3100 vaccine.

Given the challenging nature of anal HSIL, an immunotherapeutic approach such as VGX-3100 would represent a significant advancement in its management. VGX-3100 is tolerable, immunogenic and has a therapeutic effect upon HPV-16/18-associated anal HSIL. In addition, detection of HPV-16/18 from anal swabs and tissue is highly concordant (data not shown) and may offer a less invasive screening strategy to detect anal lesions, which could simplify screening and monitoring.

### Example 2: Sequences

| HPV genotype 16 consensus E6-E7 DNA sequence | |
|---|---|
| (SEQ ID NO: 1) | |
| atggactg gacctggatc ctgttcctgg tggccgccgc cacacgggtg | |
| cacagcttcc aggaccccca ggagagcggc agaaagctgc ctcagctgtg taccgagctg | |
| cagaccacca tccacgacat catcctggag tgtgtgtact gtaagcagca gctgctgagg | |
| agagaggtgt acgaccggga cctgtgtatc gtgtacaggg acggcaatcc ctacgccgtg | |
| tgtgacaagt gcctgaagtt ctacagcaag atcagcgagt accggcacta ctgctacagc | |
| ctgtacggca ccaccctgga gcagcagtac aacaagcccc tgtgtgacct gctgatccgg | |
| tgtatcaact gccagaagcc cctgcagaga cacctggaca agaagcagcg gttccacaac | |
| atcaggggca gatggaccgg cagatgtatg agctgctgcc ggagcagcag aaccagaagg | |
| gagacccagc tgagaggccg gaagagaaga agccacggcg atacccccac cctgcacgag | |
| tacatgctgg acctgcagcc tgagaccacc gatctgtacg gctacggcca gctgaatgac | |
| agcagcgagg aggaggatga gatcgacggc cctgccggcc aggccgagcc cgacagagcc | |
| cactacaaca tcgtgacctt ttgctgtaag tgtgacagca ccctgagact gtgcgtgcag | |
| agcacccacg tggacatcag aaccctggag gatctgctga tgggcaccct gggcatcgtg | |
| tgtcccatct gctcccagaa accctgatga | |
| HPV genotype 16 consensus E6-E7 protein sequence | |
| (SEQ ID NO: 2) | |
| Met Asp Trp Thr Trp Ile Leu Phe Leu Val Ala Ala Ala Thr Arg Val | |
| His Ser Phe Gln Asp Pro Gln Glu Ser Gly Arg Lys Leu Pro Gln Leu | |
| Cys Thr Glu Leu Gln Thr Thr Ile His Asp Ile Ile Leu Glu Cys Val | |
| Tyr Cys Lys Gln Gln Leu Leu Arg Arg Glu Val Tyr Asp Arg Asp Leu | |
| Cys Ile Val Tyr Arg Asp Gly Asn Pro Tyr Ala Val Cys Asp Lys Cys | |
| Leu Lys Phe Tyr Ser Lys Ile Ser Glu Tyr Arg His Tyr Cys Tyr Ser | |
| Leu Tyr Gly Thr Thr Leu Glu Gln Gln Tyr Asn Lys Pro Leu Cys Asp | |
| Leu Leu Ile Arg Cys Ile Asn Cys Gln Lys Pro Leu Gln Arg His Leu | |
| Asp Lys Lys Gln Arg Phe His Asn Ile Arg Gly Arg Trp Thr Gly Arg | |
| Cys Met Ser Cys Cys Arg Ser Ser Arg Thr Arg Arg Glu Thr Gln Leu | |
| Arg Gly Arg Lys Arg Arg Ser His Gly Asp Thr Pro Thr Leu His Glu | |
| Tyr Met Leu Asp Leu Gln Pro Glu Thr Thr Asp Leu Tyr Gly Tyr Gly | |
| Gln Leu Asn Asp Ser Ser Glu Glu Glu Asp Glu Ile Asp Gly Pro Ala | |
| Gly Gln Ala Glu Pro Asp Arg Ala His Tyr Asn Ile Val Thr Phe Cys | |
| Cys Lys Cys Asp Ser Thr Leu Arg Leu Cys Val Gln Ser Thr His Val | |
| Asp Ile Arg Thr Leu Glu Asp Leu Leu Met Gly Thr Leu Gly Ile Val | |
| Cys Pro Ile Cys Ser Gln Lys Pro | |
| HPV16 6E immunodominant epitope | |
| (SEQ ID NO: 3) | |
| Leu Cys Ile Val Tyr Arg Asp Gly Asn Pro Tyr Ala Val Cys Asp | |
| HPV16 E7 immunodominant epitope | |
| (SEQ ID NO: 4) | |
| Ala Glu Pro Asp Arg Ala His Tyr Asn Ile Val Thr Phe Cys Cys | |
| HPV16 E6 consensus sequence | |
| (SEQ ID NO: 5) | |
| Phe Gln Asp Pro Gln Glu Ser Gly Arg Lys Leu Pro Gln Leu Cys Thr | |
| Glu Leu Gln Thr Thr Ile His Asp Ile Ile Leu Glu Cys Val Tyr Cys | |
| Lys Gln Gln Leu Leu Arg Arg Glu Val Tyr Asp Arg Asp Leu Cys Ile | |
| Val Tyr Arg Asp Gly Asn Pro Tyr Ala Val Cys Asp Lys Cys Leu Lys | |
| Phe Tyr Ser Lys Ile Ser Glu Tyr Arg His Tyr Cys Tyr Ser Leu Tyr | |
| Gly Thr Thr Leu Glu Gln Gln Tyr Asn Lys Pro Leu Cys Asp Leu Leu | |
| Ile Arg Cys Ile Asn Cys Gln Lys Pro Leu Gln Arg His Leu Asp Lys | |
| Lys Gln Arg Phe His Asn Ile Arg Gly Arg Trp Thr Gly Arg Cys Met | |
| Ser Cys Cys Arg Ser Ser Arg Thr Arg Arg Glu Thr Gln Leu | |
| HPV16 E7 consensus sequence | |
| (SEQ ID NO: 6) | |
| His Gly Asp Thr Pro Thr Leu His Glu Tyr Met Leu Asp Leu Gln Pro | |
| Glu Thr Thr Asp Leu Tyr Gly Tyr Gly Gln Leu Asn Asp Ser Ser Glu | |
| Glu Glu Asp Glu Ile Asp Gly Pro Ala Gly Gln Ala Glu Pro Asp Arg | |
| Ala His Tyr Asn Ile Val Thr Phe Cys Cys Lys Cys Asp Ser Thr Leu | |
| Arg Leu Cys Val Gln Ser Thr His Val Asp Ile Arg Thr Leu Glu Asp | |
| Leu Leu Met Gly Thr Leu Gly Ile Val Cys Pro Ile Cys Ser Gln Lys | |
| Pro | |
| IgE Leader Sequence | |
| (SEQ ID NO: 7) | |
| Met Asp Trp Thr Trp Ile Leu Phe Leu Val Ala Ala Ala Thr Arg Val | |
| His Ser | |
| Proteolytic Cleavage Sequence | |
| (SEQ ID NO: 8) | |
| Arg Gly Arg Lys Arg Arg Ser | |
| HPV18 Consensus Sequence encoding E6 and E7 | |
| (SEQ ID NO: 9) | |
| gccagattcg aggaccccac caggagcggc tacaagctgc ccgatctgtg taccgagctg | 60 |
| aacaccagcc tgcaggacat cgagatcacc tgtgtgtact gtaagaccgt gctggagctg | 120 |
| accgaggtgt tcgagaagga cctgttcgtg gtgtacaggg acagcatccc ccacgccgcc | 180 |
| tgccacaagt gtatcgactt ctacagccgg atccgggagc tgagacacta cagcgacagc | 240 |
| gtgtacggcg ataccctgga gaagctgacc aacaccggcc tgtacaacct gctgatccgg | 300 |
| tgcctgagat gccagaagcc cctgctgaga cacctgaacg agaagcggcg gttccacaac | 360 |
| atcgccggcc actacagagg ccagtgccac agctgctgta acagggccag gcaggagaga | 420 |
| ctgcagcgga gaagagagac ccaggtgagg ggcaggaaga gaagaagcca cggccccaag | 480 |
| gccaccctgc aggatatcgt gctgcacctg gagccccaga atgagatccc cgtggatctg | 540 |
| ctgggccacg gccagctgtc cgacagcgag gaggagaacg acgagatcga cggcgtgaat | 600 |
| caccagcacc tgcctgccag aagagccgag cctcagaggc acaccatgct gtgtatgtgc | 660 |
| tgtaagtgtg aggcccggat cgaactggtg gtggagagca gcgccgacga cctgagagcc | 720 |
| ttccagcagc tgttcctgaa caccctgagc ttcgtgtgtc cttggtgtgc cagccagcag | 780 |
| tga | 783 |
| HPV18 Consensus HPV peptide sequence with E6 and E7 | |
| (SEQ ID NO: 10) | |
| Ala Arg Phe Glu Asp Pro Thr Arg Ser Gly Tyr Lys Leu Pro Asp Leu | |
| Cys Thr Glu Leu Asn Thr Ser Leu Gln Asp Ile Glu Ile Thr Cys Val | |
| Tyr Cys Lys Thr Val Leu Glu Leu Thr Glu Val Phe Glu Lys Asp Leu | |
| Phe Val Val Tyr Arg Asp Ser Ile Pro His Ala Ala Cys His Lys Cys | |
| Ile Asp Phe Tyr Ser Arg Ile Arg Glu Leu Arg His Tyr Ser Asp Ser | |
| Val Tyr Gly Asp Thr Leu Glu Lys Leu Thr Asn Thr Gly Leu Tyr Asn | |
| Leu Leu Ile Arg Cys Leu Arg Cys Gln Lys Pro Leu Leu Arg His Leu | |
| Asn Glu Lys Arg Arg Phe His Asn Ile Ala Gly His Tyr Arg Gly Gln | |
| Cys His Ser Cys Cys Asn Arg Ala Arg Gln Glu Arg Leu Gln Arg Arg | |
| Arg Glu Thr Gln Val Arg Gly Arg Lys Arg Arg Ser His Gly Pro Lys | |
| Ala Thr Leu Gln Asp Ile Val Leu His Leu Glu Pro Gln Asn Glu Ile | |
| Pro Val Asp Leu Leu Gly His Gly Gln Leu Ser Asp Ser Glu Glu Glu | |
| Asn Asp Glu Ile Asp Gly Val Asn His Gln His Leu Pro Ala Arg Arg | |
| Ala Glu Pro Gln Arg His Thr Met Leu Cys Met Cys Cys Lys Cys Glu | |
| Ala Arg Ile Glu Leu Val Val Glu Ser Ser Ala Asp Asp Leu Arg Ala | |
| Phe Gln Gln Leu Phe Leu Asn Thr Leu Ser Phe Val Cys Pro Trp Cys | |
| Ala Ser Gln Gln | |
| IgE Leader DNA sequence | |
| (SEQ ID NO: 11) | |
| atggactgga cctggatcct gttcctggtg gccgctgcca cacgggtgca cagc | 54 |
| IgE leader peptide sequence | |
| (SEQ ID NO: 12) | |
| Met Asp Trp Thr Trp Ile Leu Phe Leu Val Ala Ala Ala Thr Arg Val | |
| His Ser | |
| HPV18 Consensus Sequence encoding E6 and E7; include IgE leader | |
| (SEQ ID NO: 13) | |
| atggactgga cctggatcct gttcctggtg gccgctgcca cacgggtgca cagcgccaga | 60 |
| ttcgaggacc ccaccaggag cggctacaag ctgcccgatc tgtgtaccga gctgaacacc | 120 |
| agcctgcagg acatcgagat cacctgtgtg tactgtaaga ccgtgctgga gctgaccgag | 180 |
| gtgttcgaga aggacctgtt cgtggtgtac agggacagca tcccccacgc cgcctgccac | 240 |
| aagtgtatcg acttctacag ccggatccgg gagctgagac actacagcga cagcgtgtac | 300 |
| ggcgataccc tggagaagct gaccaacacc ggcctgtaca acctgctgat ccggtgcctg | 360 |
| agatgccaga agcccctgct gagacacctg aacgagaagc ggcggttcca caacatcgcc | 420 |
| ggccactaca gaggccagtg ccacagctgc tgtaacaggg ccaggcagga gagactgcag | 480 |
| cggagaagag agacccaggt gaggggcagg aagagaagaa gccacggccc caaggccacc | 540 |
| ctgcaggata tcgtgctgca cctggagccc cagaatgaga tccccgtgga tctgctgggc | 600 |
| cacggccagc tgtccgacag cgaggaggag aacgacgaga tcgacggcgt gaatcaccag | 660 |
| cacctgcctg ccagaagagc cgagcctcag aggcacacca tgctgtgtat gtgctgtaag | 720 |
| tgtgaggccc ggatcgaact ggtggtggag agcagcgccg acgacctgag agccttccag | 780 |
| cagctgttcc tgaacaccct gagcttcgtg tgtccttggt gtgccagcca gcagtga | 837 |
| HPV18 Consensus HPV peptide sequence with E6 and E7; | |
| including IgE leader | |
| (SEQ ID NO: 14) | |
| Met Asp Trp Thr Trp Ile Leu Phe Leu Val Ala Ala Ala Thr Arg Val | |
| His Ser Ala Arg Phe Glu Asp Pro Thr Arg Ser Gly Tyr Lys Leu Pro | |
| Asp Leu Cys Thr Glu Leu Asn Thr Ser Leu Gln Asp Ile Glu Ile Thr | |
| Cys Val Tyr Cys Lys Thr Val Leu Glu Leu Thr Glu Val Phe Glu Lys | |
| Asp Leu Phe Val Val Tyr Arg Asp Ser Ile Pro His Ala Ala Cys His | |
| Lys Cys Ile Asp Phe Tyr Ser Arg Ile Arg Glu Leu Arg His Tyr Ser | |
| Asp Ser Val Tyr Gly Asp Thr Leu Glu Lys Leu Thr Asn Thr Gly Leu | |
| Tyr Asn Leu Leu Ile Arg Cys Leu Arg Cys Gln Lys Pro Leu Leu Arg | |
| His Leu Asn Glu Lys Arg Arg Phe His Asn Ile Ala Gly His Tyr Arg | |
| Gly Gln Cys His Ser Cys Cys Asn Arg Ala Arg Gln Glu Arg Leu Gln | |
| Arg Arg Arg Glu Thr Gln Val Arg Gly Arg Lys Arg Arg Ser His Gly | |
| Pro Lys Ala Thr Leu Gln Asp Ile Val Leu His Leu Glu Pro Gln Asn | |
| Glu Ile Pro Val Asp Leu Leu Gly His Gly Gln Leu Ser Asp Ser Glu | |
| Glu Glu Asn Asp Glu Ile Asp Gly Val Asn His Gln His Leu Pro Ala | |
| Arg Arg Ala Glu Pro Gln Arg His Thr Met Leu Cys Met Cys Cys Lys | |
| Cys Glu Ala Arg Ile Glu Leu Val Val Glu Ser Ser Ala Asp Asp Leu | |
| Arg Ala Phe Gln Gln Leu Phe Leu Asn Thr Leu Ser Phe Val Cys Pro | |
| Trp Cys Ala Ser Gln Gln | |
| pGX3002 plasmid with consensus and IgE leader | |
| (SEQ ID NO: 15) | |
| gctgcttcgc gatgtacggg ccagatatac gcgttgacat tgattattga ctagttatta | 60 |
| atagtaatca attacggggt cattagttca tagcccatat atggagttcc gcgttacata | 120 |
| acttacggta aatggcccgc ctggctgacc gcccaacgac ccccgcccat tgacgtcaat | 180 |
| aatgacgtat gttcccatag taacgccaat agggactttc cattgacgtc aatgggtgga | 240 |
| gtatttacgg taaactgccc acttggcagt acatcaagtg tatcatatgc caagtacgcc | 300 |
| ccctattgac gtcaatgacg gtaaatggcc cgcctggcat tatgcccagt acatgacctt | 360 |
| atgggacttt cctacttggc agtacatcta cgtattagtc atcgctatta ccatggtgat | 420 |
| gcggttttgg cagtacatca atgggcgtgg atagcggttt gactcacggg gatttccaag | 480 |
| tctccacccc attgacgtca atgggagttt gttttggcac caaaatcaac gggactttcc | 540 |
| aaaatgtcgt aacaactccg ccccattgac gcaaatgggc ggtaggcgtg tacggtggga | 600 |
| ggtctatata agcagagctc tctggctaac tagagaaccc actgcttact ggcttatcga | 660 |
| aattaatacg actcactata gggagaccca agctggctag cgtttaaact taagcttggt | 720 |
| accgagctcg gatccactag tccagtgtgg tggaattcgc caccatggac tggacctgga | 780 |
| tcctgttcct ggtggccgct gccacacggg tgcacagcgc cagattcgag gaccccacca | 840 |
| ggagcggcta caagctgccc gatctgtgta ccgagctgaa caccagcctg caggacatcg | 900 |
| agatcacctg tgtgtactgt aagaccgtgc tggagctgac cgaggtgttc gagaaggacc | 960 |
| tgttcgtggt gtacagggac agcatccccc acgccgcctg ccacaagtgt atcgacttct | 1020 |
| acagccggat ccgggagctg agacactaca gcgacagcgt gtacggcgat accctggaga | 1080 |
| agctgaccaa caccggcctg tacaacctgc tgatccggtg cctgagatgc cagaagcccc | 1140 |
| tgctgagaca cctgaacgag aagcggcggt tccacaacat cgccggccac tacagaggcc | 1200 |
| agtgccacag ctgctgtaac agggccaggc aggagagact gcagcggaga agagagaccc | 1260 |
| aggtgagggg caggaagaga agaagccacg gccccaaggc caccctgcag gatatcgtgc | 1320 |
| tgcacctgga gccccagaat gagatccccg tggatctgct gggccacggc cagctgtccg | 1380 |
| acagcgagga ggagaacgac gagatcgacg gcgtgaatca ccagcacctg cctgccagaa | 1440 |
| gagccgagcc tcagaggcac accatgctgt gtatgtgctg taagtgtgag gcccggatcg | 1500 |
| aactggtggt ggagagcagc gccgacgacc tgagagcctt ccagcagctg ttcctgaaca | 1560 |
| ccctgagctt cgtgtgtcct tggtgtgcca gccagcagtg atgagcggcc gctcgagtct | 1620 |
| agagggcccg tttaaacccg ctgatcagcc tcgactgtgc cttctagttg ccagccatct | 1680 |
| gttgtttgcc cctcccccgt gccttccttg accctggaag gtgccactcc cactgtcctt | 1740 |
| tcctaataaa atgaggaaat tgcatcgcat tgtctgagta ggtgtcattc tattctgggg | 1800 |
| ggtggggtgg ggcaggacag caagggggag gattgggaag acaatagcag gcatgctggg | 1860 |
| gatgcggtgg gctctatggc ttctactggg cggttttatg gacagcaagc gaaccggaat | 1920 |
| tgccagctgg ggcgccctct ggtaaggttg ggaagccctg caaagtaaac tggatggctt | 1980 |
| tcttgccgcc aaggatctga tggcgcaggg gatcaagctc tgatcaagag acaggatgag | 2040 |
| gatcgtttcg catgattgaa caagatggat tgcacgcagg ttctccggcc gcttgggtgg | 2100 |
| agaggctatt cggctatgac tgggcacaac agacaatcgg ctgctctgat gccgccgtgt | 2160 |
| tccggctgtc agcgcagggg cgcccggttc tttttgtcaa gaccgacctg tccggtgccc | 2220 |
| tgaatgaact gcaagacgag gcagcgcggc tatcgtggct ggccacgacg ggcgttcctt | 2280 |
| gcgcagctgt gctcgacgtt gtcactgaag cgggaaggga ctggctgcta ttgggcgaag | 2340 |
| tgccggggca ggatctcctg tcatctcacc ttgctcctgc cgagaaagta tccatcatgg | 2400 |
| ctgatgcaat gcggcggctg catacgcttg atccggctac ctgcccattc gaccaccaag | 2460 |
| cgaaacatcg catcgagcga gcacgtactc ggatggaagc cggtcttgtc gatcaggatg | 2520 |
| atctggacga agagcatcag gggctcgcgc cagccgaact gttcgccagg ctcaaggcga | 2580 |
| gcatgcccga cggcgaggat ctcgtcgtga cccatggcga tgcctgcttg ccgaatatca | 2640 |
| tggtggaaaa tggccgcttt tctggattca tcgactgtgg ccggctgggt gtggcggacc | 2700 |
| gctatcagga catagcgttg gctacccgtg atattgctga agagcttggc ggcgaatggg | 2760 |
| ctgaccgctt cctcgtgctt tacggtatcg ccgctcccga ttcgcagcgc atcgccttct | 2820 |
| atcgccttct tgacgagttc ttctgaatta ttaacgctta caatttcctg atgcggtatt | 2880 |
| ttctccttac gcatctgtgc ggtatttcac accgcatcag gtggcacttt tcggggaaat | 2940 |
| gtgcgcggaa cccctatttg tttatttttc taaatacatt caaatatgta tccgctcatg | 3000 |
| agacaataac cctgataaat gcttcaataa tagcacgtgc taaaacttca tttttaattt | 3060 |
| aaaaggatct aggtgaagat cctttttgat aatctcatga ccaaaatccc ttaacgtgag | 3120 |
| ttttcgttcc actgagcgtc agaccccgta gaaaagatca aaggatcttc ttgagatcct | 3180 |
| ttttttctgc gcgtaatctg ctgcttgcaa acaaaaaaac caccgctacc agcggtggtt | 3240 |
| tgtttgccgg atcaagagct accaactctt tttccgaagg taactggctt cagcagagcg | 3300 |
| cagataccaa atactgttct tctagtgtag ccgtagttag gccaccactt caagaactct | 3360 |
| gtagcaccgc ctacatacct cgctctgcta atcctgttac cagtggctgc tgccagtggc | 3420 |
| gataagtcgt gtcttaccgg gttggactca agacgatagt taccggataa ggcgcagcgg | 3480 |
| tcgggctgaa cggggggttc gtgcacacag cccagcttgg agcgaacgac ctacaccgaa | 3540 |
| ctgagatacc tacagcgtga gctatgagaa agcgccacgc ttcccgaagg gagaaaggcg | 3600 |
| gacaggtatc cggtaagcgg cagggtcgga acaggagagc gcacgaggga gcttccaggg | 3660 |
| ggaaacgcct ggtatcttta tagtcctgtc gggtttcgcc acctctgact tgagcgtcga | 3720 |
| tttttgtgat gctcgtcagg ggggcggagc ctatggaaaa acgccagcaa cgcggccttt | 3780 |
| ttacggttcc tggccttttg ctggcctttt gctcacatgt tctt | 3824 |

HPV genotype 16 consensus E6-E7 peptide sequence without IgE leader
coding sequence
(SEQ ID NO: 16)
ttcc aggaccccca ggagagcggc agaaagctgc ctcagctgtg taccgagctg
cagaccacca tccacgacat catcctggag tgtgtgtact gtaagcagca gctgctgagg
agagaggtgt acgaccggga cctgtgtatc gtgtacaggg acggcaatcc ctacgccgtg
tgtgacaagt gcctgaagtt ctacagcaag atcagcgagt accggcacta ctgctacagc
ctgtacggca ccaccctgga gcagcagtac aacaagcccc tgtgtgacct gctgatccgg
tgtatcaact gccagaagcc cctgcagaga cacctggaca agaagcagcg gttccacaac
atcaggggca gatggaccgg cagatgtatg agctgctgcc ggagcagcag aaccagaagg
gagacccagc tgagaggccg gaagagaaga agccacggcg atacccccac cctgcacgag
tacatgctgg acctgcagcc tgagaccacc gatctgtacg gctacggcca gctgaatgac
agcagcgagg aggaggatga gatcgacggc cctgccggcc aggccgagcc cgacagagcc
cactacaaca tcgtgacctt ttgctgtaag tgtgacagca ccctgagact gtgcgtgcag
agcacccacg tggacatcag aaccctggag gatctgctga tgggcaccct gggcatcgtg
tgtcccatct gctcccagaa accc
HPV genotype 16 consensus E6 nucleotide sequence
(SEQ ID NO: 17)
ttcc aggaccccca ggagagcggc agaaagctgc ctcagctgtg taccgagctg
cagaccacca tccacgacat catcctggag tgtgtgtact gtaagcagca gctgctgagg
agagaggtgt acgaccggga cctgtgtatc gtgtacaggg acggcaatcc ctacgccgtg
tgtgacaagt gcctgaagtt ctacagcaag atcagcgagt accggcacta ctgctacagc
ctgtacggca ccaccctgga gcagcagtac aacaagcccc tgtgtgacct gctgatccgg
tgtatcaact gccagaagcc cctgcagaga cacctggaca agaagcagcg gttccacaac
atcaggggca gatggaccgg cagatgtatg agctgctgcc ggagcagcag aaccagaagg
gagacccagc tg
Nucleotide sequence encoding Proteolytic Cleavage Sequence
(SEQ ID NO: 18)
agaggccg gaagagaaga agc
HPV genotype 16 consensus E7 nucleotide sequence
(SEQ ID NO: 19)
cacggcg atacccccac cctgcacgag
tacatgctgg acctgcagcc tgagaccacc gatctgtacg gctacggcca gctgaatgac
agcagcgagg aggaggatga gatcgacggc cctgccggcc aggccgagcc cgacagagcc
cactacaaca tcgtgacctt ttgctgtaag tgtgacagca ccctgagact gtgcgtgcag
agcacccacg tggacatcag aaccctggag gatctgctga tgggcaccct gggcatcgtg
tgtcccatct gctcccagaa accc
HPV genotype 16 consensus E6-E7 peptide sequence without IgE
leader sequence
(SEQ ID NO: 20)
Phe Gln Asp Pro Gln Glu Ser Gly Arg Lys Leu Pro Gln Leu
Cys Thr Glu Leu Gln Thr Thr Ile His Asp Ile Ile Leu Glu Cys Val
Tyr Cys Lys Gln Gln Leu Leu Arg Arg Glu Val Tyr Asp Arg Asp Leu
Cys Ile Val Tyr Arg Asp Gly Asn Pro Tyr Ala Val Cys Asp Lys Cys
Leu Lys Phe Tyr Ser Lys Ile Ser Glu Tyr Arg His Tyr Cys Tyr Ser
Leu Tyr Gly Thr Thr Leu Glu Gln Gln Tyr Asn Lys Pro Leu Cys Asp
Leu Leu Ile Arg Cys Ile Asn Cys Gln Lys Pro Leu Gln Arg His Leu
Asp Lys Lys Gln Arg Phe His Asn Ile Arg Gly Arg Trp Thr Gly Arg
Cys Met Ser Cys Cys Arg Ser Ser Arg Thr Arg Arg Glu Thr Gln Leu
Arg Gly Arg Lys Arg Arg Ser His Gly Asp Thr Pro Thr Leu His Glu
Tyr Met Leu Asp Leu Gln Pro Glu Thr Thr Asp Leu Tyr Gly Tyr Gly
Gln Leu Asn Asp Ser Ser Glu Glu Glu Asp Glu Ile Asp Gly Pro Ala
Gly Gln Ala Glu Pro Asp Arg Ala His Tyr Asn Ile Val Thr Phe Cys
Cys Lys Cys Asp Ser Thr Leu Arg Leu Cys Val Gln Ser Thr His Val
Asp Ile Arg Thr Leu Glu Asp Leu Leu Met Gly Thr Leu Gly Ile Val
Cys Pro Ile Cys Ser Gln Lys Pro
HPV18 Consensus E6 peptide
(SEQ ID NO: 21)
Ala Arg Phe Glu Asp Pro Thr Arg Ser Gly Tyr Lys Leu Pro
Asp Leu Cys Thr Glu Leu Asn Thr Ser Leu Gln Asp Ile Glu Ile Thr
Cys Val Tyr Cys Lys Thr Val Leu Glu Leu Thr Glu Val Phe Glu Lys
Asp Leu Phe Val Val Tyr Arg Asp Ser Ile Pro His Ala Ala Cys His
Lys Cys Ile Asp Phe Tyr Ser Arg Ile Arg Glu Leu Arg His Tyr Ser
Asp Ser Val Tyr Gly Asp Thr Leu Glu Lys Leu Thr Asn Thr Gly Leu
Tyr Asn Leu Leu Ile Arg Cys Leu Arg Cys Gln Lys Pro Leu Leu Arg
His Leu Asn Glu Lys Arg Arg Phe His Asn Ile Ala Gly His Tyr Arg
Gly Gln Cys His Ser Cys Cys Asn Arg Ala Arg Gln Glu Arg Leu Gln
Arg Arg Arg Glu Thr Gln Val
HPV18 Consensus E7 peptide
(SEQ ID NO: 22)
His Gly Pro Lys Ala Thr Leu Gln Asp Ile Val Leu His Leu Glu Pro Gln Asn
Glu Ile Pro Val Asp Leu Leu Gly His Gly Gln Leu Ser Asp Ser Glu
Glu Glu Asn Asp Glu Ile Asp Gly Val Asn His Gln His Leu Pro Ala
Arg Arg Ala Glu Pro Gln Arg His Thr Met Leu Cys Met Cys Cys Lys
Cys Glu Ala Arg Ile Glu Leu Val Val Glu Ser Ser Ala Asp Asp Leu
Arg Ala Phe Gln Gln Leu Phe Leu Asn Thr Leu Ser Phe Val Cys Pro
Trp Cys Ala Ser Gln Gln
HPV18 Consensus Sequence encoding E6
(SEQ ID NO: 23)
gccagattcg aggaccccac caggagcggc tacaagctgc ccgatctgtg taccgagctg
aacaccagcc tgcaggacat cgagatcacc tgtgtgtact gtaagaccgt gctggagctg
accgaggtgt tcgagaagga cctgttcgtg gtgtacaggg acagcatccc ccacgccgcc
tgccacaagt gtatcgactt ctacagccgg atccgggagc tgagacacta cagcgacagc
gtgtacggcg ataccctgga gaagctgacc aacaccggcc tgtacaacct gctgatccgg
tgcctgagat gccagaagcc cctgctgaga cacctgaacg agaagcggcg gttccacaac
atcgccggcc actacagagg ccagtgccac agctgctgta acagggccag gcaggagaga
ctgcagcgga gaagagagac ccaggtg
HPV18 Consensus Sequence encoding E7
(SEQ ID NO: 24)
ca cggccccaag gccaccctgc aggatatcgt gctgcacctg gagccccaga atgagatccc
cgtggatctg ctgggccacg gccagctgtc cgacagcgag gaggagaacg acgagatcga
cggcgtgaat caccagcacc tgcctgccag aagagccgag cctcagaggc acaccatgct
gtgtatgtgc tgtaagtgtg aggcccggat cgaactggtg gtggagagca gcgccgacga
cctgagagcc ttccagcagc tgttcctgaa caccctgagc ttcgtgtgtc cttggtgtgc
cagccagcag

The examples and embodiments described herein are for illustrative purposes only and various modifications or changes suggested to persons skilled in the art are to be included within the spirit and purview of this application and scope of the appended claims.

1. A method for treating or preventing anal high-grade squamous intraepithelial lesion (HSIL) in an individual comprising administering to the individual a composition comprising at least one nucleic acid molecule encoding at least one selected from the group consisting of: a human papillomavirus (HPV) 16 antigen and an HPV18 antigen.

2. The method of claim 1, wherein the HPV16 antigen is an HPV16 E6-E7 fusion antigen.

3. The method of claim 1, wherein the HPV16 antigen comprises the amino acid sequence of SEQ ID NO: 5 and the amino acid sequence of SEQ ID NO: 6.

4. The method of claim 1, wherein the HPV16 antigen is encoded by a nucleic acid molecule comprising the nucleotide sequence of SEQ ID NO: 17 and the nucleotide sequence of SEQ ID NO: 19.

5. The method of claim 1, wherein the HPV18 antigen is an HPV18 E6-E7 fusion antigen.

6. The method of claim 1, wherein the HPV18 antigen comprises the amino acid sequence of SEQ ID NO: 21 and the amino acid sequence of SEQ ID NO: 22.

7. The method of claim 1, wherein the composition comprises a nucleic acid molecule comprising a nucleotide sequence encoding an HPV16 antigen and a nucleic acid molecule comprising a nucleotide sequence encoding an HPV18 antigen.

8. The method of claim 1, wherein the nucleic acid molecule comprises one or more nucleotide sequences selected from the group consisting of:
a nucleotide sequence that encodes SEQ ID NO:2;
a nucleotide sequence that is at least 95% homologous to a nucleotide sequence that encodes SEQ ID NO:2;
a fragment of a nucleotide sequence that encodes SEQ ID NO:2;
a nucleotide sequence that is at least 95% homologous to a fragment of a nucleotide sequence that encodes SEQ ID NO:2.

9. The method of claim 8, wherein the nucleic acid molecule comprises a nucleotide sequence that is at least 98% homologous to a nucleotide sequence that encodes SEQ ID NO:2.

10. The method of claim 8, wherein the nucleic acid molecule comprises a nucleotide sequence that is at least 99% homologous to a nucleotide sequence that encodes SEQ ID NO:2.

11. The method of claim 1, wherein the nucleic acid molecule comprises one or more nucleotide sequences selected from the group consisting of:
a nucleotide sequence comprising nucleotides 19-795 of SEQ ID NO:1;
a nucleotide sequence comprising nucleotides 1-795 of SEQ ID NO:1;
a nucleotide sequence comprising SEQ ID NO:1;
a nucleotide sequence that is at least 95% homologous SEQ ID NO:1;
a fragment of SEQ ID NO:1;
a nucleotide sequence that is at least 95% homologous to a fragment of SEQ ID NO:1.

12. The method of claim 11, wherein the nucleic acid molecule comprises a nucleotide sequence that is at least 98% homologous to SEQ ID NO:1.

13. The method of claim 11, wherein the nucleic acid molecule comprises a nucleotide sequence that is at least 99% homologous to SEQ ID NO:1.

14. The method of claim 1, wherein the HPV18 antigen is encoded by a nucleic acid molecule comprising the nucleotide sequence of SEQ ID NO: 23 and the nucleotide sequence of SEQ ID NO: 24.

15. The method of claim 5, wherein the nucleic acid molecule comprises one or more nucleotide sequences selected from the group consisting of:
a nucleotide sequence that encodes SEQ ID NO:10;
a nucleotide sequence that is at least 95% homologous to a nucleotide sequence that encodes SEQ ID NO:10;
a fragment of a nucleotide sequence that encodes SEQ ID NO:10;
a nucleotide sequence that is at least 95% homologous to a fragment of a nucleotide sequence that encodes SEQ ID NO:10.

16. The method of claim 15, wherein the nucleic acid molecule comprises a nucleotide sequence that is at least 98% homologous to a nucleotide sequence that encodes SEQ ID NO:10.

17. The method of claim 15, wherein the nucleic acid molecule comprises a nucleotide sequence that is at least 99% homologous to a nucleotide sequence that encodes SEQ ID NO:10.

18. The method of claim 15, wherein the nucleic acid molecule encoding the HPV16 E6-E7 fusion antigen further comprises a nucleotide sequence encoding a leader sequence, wherein the nucleotide sequence encoding the HPV18 E6-E7 fusion antigen further comprises a nucleotide sequence encoding a leader sequence, or wherein the nucleotide sequence encoding the HPV16 E6-E7 fusion antigen further comprises a nucleotide sequence encoding a leader sequence and the nucleotide sequence encoding the HPV18 E6-E7 fusion antigen further comprises a nucleotide sequence encoding a leader sequence.

19. The method of claim 5, wherein the nucleic acid molecule comprises one or more nucleotide sequences selected from the group consisting of:
a nucleotide sequence comprising nucleotides 1-780 of SEQ ID NO:9;
a nucleotide sequence comprising SEQ ID NO:9;
a nucleotide sequence that is at least 95% homologous SEQ ID NO:9;
a fragment of SEQ ID NO:9;
a nucleotide sequence that is at least 95% homologous to a fragment of SEQ ID NO:9.

20. The method of claim 19, wherein the nucleic acid molecule comprises a nucleotide sequence that is at least 98% homologous to SEQ ID NO:9.

21. The method of claim 19, wherein the nucleic acid molecule comprises a nucleotide sequence that is at least 99% homologous to SEQ ID NO:9.

22. The method of claim 1, wherein the at least one nucleic acid molecule comprises at least one plasmid.

23. The method of claim 1, wherein the composition is a pharmaceutical composition.

24. The method of claim 1, further comprising administering to the individual a composition comprising an adjuvant.

25. The method of claim 7, comprising administering to the individual a nucleotide sequence encoding an HPV16 E6-E7 fusion antigen and a nucleotide sequence encoding an HPV18 E6-E7 fusion antigen;
wherein the nucleotide sequence encoding the HPV16 E6-E7 fusion antigen is selected from the group consisting of: a nucleotide sequence that encodes SEQ ID NO:2; a nucleotide sequence that is at least 95% homologous to a nucleotide sequence that encodes SEQ ID NO:2; a fragment of a nucleotide sequence that encodes SEQ ID NO:2; and a nucleotide sequence that is at least 95% homologous to a fragment of a nucleotide sequence that encodes SEQ ID NO:2; and
wherein the nucleotide sequence encoding the HPV18 E6-E7 fusion antigen is selected from the group consisting of: a nucleotide sequence that encodes SEQ ID NO:10; a nucleotide sequence that is at least 95% homologous to a nucleotide sequence that encodes SEQ ID NO:10; a fragment of a nucleotide sequence that encodes SEQ ID NO:10; and a nucleotide sequence that is at least 95% homologous to a fragment of a nucleotide sequence that encodes SEQ ID NO:10.

26. The method of claim 1, wherein administering said nucleic acid molecule to the individual comprises electroporation.

27. The method of claim 1 wherein the composition is administered intramuscularly.

28. The method of claim 1, wherein the composition comprises VGX-3100 or a biosimilar thereof.

29. The method of claim 1, wherein the composition is administered in three or four doses.

30. The method of claim 29, wherein the second dose of the composition is administered about four weeks after the first dose.

31. The method of claim 29, wherein the third dose is administered about twelve weeks after the first dose.

32. The method of claim 29, wherein the fourth dose is administered about 40 weeks after the first dose.

33. The method of claim 1, wherein the HSIL is anal or anal/peri-anal HSIL.

34. The method of claim 18, wherein the leader sequence comprises the amino acid sequence of SEQ ID NO: 7.

35. The method of claim 18, wherein the leader sequence is encoded by a nucleic acid molecule comprising the nucleotide sequence of SEQ ID NO: 11.
